(19) United States
(12) Patent Application Publication
(10) Pub. No.: US 2023/0233448 A1
(43) Pub. Date: Jul. 27, 2023

(54) TOPICAL COMPOSITIONS FOR TREATING OR PREVENTING SCARS AND METHODS FOR THEIR USE

(71) Applicant: Alderwood Surgical Center, LLC, Lynnwood, WA (US)

(72) Inventor: Javad Sajan, Bellevue, WA (US)

(21) Appl. No.: 18/153,514

(22) Filed: Jan. 12, 2023

Related U.S. Application Data
(60) Provisional application No. 63/298,878, filed on Jan. 12, 2022.

(51) Int. Cl. A61K 8/9794; A61Q 19/10; A61K 8/92; A61K 8/67; A61K 8/9789; A61K 8/891

(52) U.S. Cl. CPC A61K 8/9794; A61K 8/671; A61K 8/678; A61K 8/891; A61K 8/925; A61K 8/9789; A61Q 19/10; A61K 2800/884; A61K 2800/5922

(57) ABSTRACT

The present technology includes a set of topical compositions used in treating wounds or scars in accordance with the methods and protocols described herein. The set of topical compositions can include (i) a cleansing composition comprising polydimethylsiloxane (silicone) and Rosmarinus officinalis (rosemary) flower extract; (ii) a first treatment composition comprising Aloe barbadensis (aloe) leaf juice, Macadamia temifolia (macadamia) seed oil, and panthenol (vitamin B5); (iii) a second treatment composition comprising Aloe barbadensis (aloe) leaf extract, sodium hyaluronate, and allantoin; (iv) a third treatment composition comprising snail mucin extract; (v) a fourth treatment composition comprising tocopherol; and (v) a fifth treatment composition comprising retinol.

### PRIORITY CLAIM

This application claims the benefit of U.S. Provisional Pat. Application No. 63/298,878, filed on Jan. 12, 2022, which is incorporated herein by reference in its entirety.

### TECHNICAL FIELD

The present technology generally relates to topical compositions, kits, methods, and protocols for healing and preventing scars and/or wounds.

### BACKGROUND

Scars form as part of the body’s healing process after a subject’s skin has been cut or damaged. The skin repairs itself by growing new tissue to pull together the wound and fill in any gaps caused by the injury. Scar tissue is made primarily of collagen. Not all scars require treatment, but some are large and painful and/or unsightly. Many treatments exist for alleviating symptoms for reducing the appearance of unwanted scars, but few—if any—are effective in achieving desired results. As such, there is a need for effective and convenient alternatives to treat existing scars or to prevent scars from forming after an injury or surgery.

### SUMMARY

The present technology is generally directed to a set of topical compositions used in preventing or treating wounds or scars. In some embodiments, the set of topical compositions comprises a cleansing composition and at least one treatment composition. Application of the set of topical compositions can reduce or prevent the appearance of scars or wounds. Application of the set of topical compositions can also reduce the size or pigmentation of scars or wounds.

In some embodiments, the present technology includes a set of topical compositions comprising a cleansing composition and at least one treatment composition comprising at least one component selected from the group consisting of Aloe barbadensis leaf juice, snail mucin extract, tocopherol, and retinol. In certain embodiments, the cleansing composition comprises polydimethylsiloxane (silicone) and Rosmarinus officinalis (rosemary) flower extract, and the first treatment composition comprises Aloe barbadensis (aloe or aloe vera) leaf juice, Macadamia ternifolia (macadamia) seed oil, and panthenol (vitamin B5).

In some embodiments, the set of topical compositions further comprises a second treatment composition which comprises Aloe barbadensis leaf extract, sodium hyaluronate, and allantoin, a third treatment composition which comprises snail mucin extract, a fourth treatment composition which comprises tocopherol, and a fifth treatment composition which comprises retinol. In certain embodiments, the polydimethylsiloxane is present in the cleansing composition in an amount of about 5% by weight, the rosemary flower extract in the cleansing composition is present in the cleansing composition in an amount of about 15% by weight, the aloe leaf juice is present in the first treatment composition in an amount of about 15% by weight, the macadamia seed oil is present in the first treatment composition in an amount of about 1.5% by weight, the panthenol (vitamin B5) is present in the first treatment composition in an amount of about 2% by weight, the aloe leaf extract is present in the second treatment composition in an amount of about 3% by weight, the Sodium Hyaluronate is present in the second treatment composition in an amount of about 3% by weight, the allantoin is present in the second treatment composition in an amount of about 2% by weight, the snail mucin extract is present in the third treatment composition in an amount of about 5% by weight, the tocopherol is present in the fourth cleansing composition in an amount of about 5% by weight, the retinol is supplied at a strength of 2.5% and is present in the fifth treatment composition in an amount of about 2.5% by weight, or a combination thereof.

In some aspects, the cleansing composition further comprises polydimethylsiloxane (silicone), Cocos Nucifera (Coconut) Oil, Europaea (Olive) Fruit Oil, Helianthus Annuus (Sunflower) Seed Oil, Aloe Barbadenis (Aloe) Leaf Juice, Citric Acid, Polydimethylsiloxane (Silicone), Butyrospermum Parkii (Shea) Butter, Rosmarinus Officinalis (Rosemary) Oil, Rosmarinus Officinalis (Rosemary) Flower Extract, or any combination thereof.

In some aspects, the first treatment composition further comprises Aloe Barbadensis (Aloe) Leaf Juice, Aqua (Deionized Water), Tocopheryl Acetate (Vitamin E), Helianthus Annuus (Sunflower) Seed Oil, Glyceryl Steswax, Potassium Stearate, Simmondsia Chinensis (Jojoba) Seed Oil, Butyrospermum Parkii (Shea) Butter, Calophyllum Inophyllum (Tamanu) Seed Oil, Macadamia Ternifolia (Macadamia) Seed Oil, Aleurites Moluccana (Kukui) Seed Oil, Persea Gratissima (Avocado) Oil, Borago Officinalis (Borage) Seed Oil, Linum Usitatissimum (Flax) Seed Oil, Oenothera Biennis (Evening Primrose) Oil, Rosa Canina (Rosehip) Fruit Oil, Panthenol (Vitamin B5), Camellia Sinensis (Green Tea) Leaf Extract, Morinda Citrifolia (Noni) Extract, Passiflora Incarnata (Passionflower) Extract, Calendula Officinalis (Calendula) Extract, Chamomilla Recutita (Chamomile) Extract, Lavandula Angustifolia (Lavender) Extract, Arnica Montana (Arnica) Extract, Centella Asiatica (Gotu Kola) Extract, Equisetum Arvense (Horsetail) Extract, Geranium Maculatum (Wild Geranium) Extract, Taraxacum Officinale (Dandelion) Extract, Stearic Acid, Xanthan Gum, Sodium Carbomer, Pentylene Glycol, or any combination thereof.

In some aspects, the second treatment composition further comprises Aqua (Deionized Water), Glycerin, Propylene Glycol, Ethylhexyl Palmitate (Palm Oil Derivate), Sorbitan Olivate, Caprylic/Capric Triglyceride, Paraffinum Liquidum, Squalane, Dimethicone, Isohexadecane, Hydrogenated Coco-glycerides (Coconut Oil), Polysorbate 60, Hydroxyethyl Acrylate/Sodium Acryloyldimethyl Taurate Copolymer, Sodium Acrylate/Sodium Acryloyldimethyl Taurate Copolymer, Althaea Officinalis (Marsh-Mallow) Root Extract, Beta-Glucan, Aloe Barbadensis (Aloe) Leaf Extract, Sodium Hyaluronate, Allantoin, Ethylhexylglycerin, or any combination thereof.

In some aspects, the third treatment composition further comprises Aqua (Deionized Water), Glycerol, Snail Mucin Extract, Viola Tricolor (Wild Pansy) Extract, Aloe Barbadensis (Aloe) Leaf Juice, Butyrospermum Parkii (Shea Tree), Oryza Sativa (Rice) Germ, Rosa Gallica (French Rose) Extract, Caprylic / Capric Triglyceride, Squalane, Betaine, Dimethicone, Xanthan Gum, Tocopherol (Vitamin E), Laurocapram, Sodium Hyaluronate, or any combination thereof.

In some aspects, the fourth treatment composition further comprises the contents of Aqua (Deionized Water), Theobroma Cacao (Cocoa) Butter, Butyrospermum Parkii (Shea) Butter, Cocos Nucifera (Coconut) Oil, Glycerin, Tocopherol (Vitamin E), Polydimethylsiloxane (Silicone), Xanthan Gum, Salix Alba (White Willow) Bark Extract, Rosmarinus Officianalis (Rosemary) Leaf Extract, Azadirachta Indica (Neem) Seed Oil, or any combination thereof.

In some aspects, the fifth treatment composition further comprises the contents of Aqua (Deionized Water), Aloe Barbadensis (Aloe) Extract, Helianthus Annuus (Sunflower) Oil, Glyceryl Stearate, Stearic Acid, Cassia Angustifolia (Senna) Seed, Vegetable Glycerin, Retinol, Simmondsia Chinensis (Jojoba) Oil, Camellia Sinensis (Green Tea) Leaf Extract, Polydimethylsiloxane (Silicone), Resina Propoli (Beeswax Acid), Butyrospermum Parkii (Shea) Butter, Panthenol (Vitamin B5), Tocopherol (Vitamin E), Xanthan Gum, Phospholipids (Sunflower Lecithin), Pentylene Glycol, Polysorbate 20, Potassium Sorbate, Ethylhexyl Glycerin, or any combination thereof.

In some embodiments, the present technology includes a method for treating or preventing a scar or wound. In some embodiments, the method comprises applying a cleansing composition followed by a first treatment composition to a wound or scar at least once daily for a first period of time. In some embodiments, the cleansing composition comprises silicone and rosemary flower extract, and the first treatment composition comprises aloe leaf juice, macadamia seed oil, and vitamin B5. After the first period of time, the method further comprises applying a second treatment composition to the wound or scar at least once daily for a second period of time. In some embodiments, the second treatment composition comprises aloe leaf extract, sodium hyaluronate, and allantoin. After the second period of time, the method further comprises applying a third treatment composition to the wound or scar at least once daily for a third period of time, wherein the third treatment composition comprises snail mucin extract. After the third period of time, the method further comprises applying a fourth treatment composition to the wound or scar at least once daily for a fourth period of time. In some embodiments, the fourth treatment composition comprises tocopherol. After the fourth period of time, the method further comprises applying a fifth treatment composition to the wound or scar at least once daily for a fifth period of time. In some embodiments, the fifth treatment composition comprises retinol.

In certain embodiments, the first period of time is about a month. In certain embodiments, the second period of time is about 2 or 3 months. In certain embodiments, the third period of time is about 2 or 3 months. In certain embodiments, the fourth period of time is about 3 or 4 months. In certain embodiments, the fifth period of time is about 2 or 3 months. In certain embodiments, the first period of time begins about a week to about six months after the wound occurs or the scar begins to form. In certain embodiments, the cleansing composition and each of the treatment compositions are applied topically to the wound or the scar. In certain embodiments, each of the treatment compositions are applied twice daily.

In some embodiments, the present technology includes a kit comprising a set of topical compositions comprising a cleansing composition and at least one treatment composition comprising at least one component selected from the group consisting of Aloe barbadensis leaf juice, snail mucin extract, tocopherol, and retinol; a container that holds a container for containing the cleansing composition; a container for containing each treatment composition; and an instructional insert card.

The foregoing paragraphs have been provided by way of general introduction and are not intended to limit the scope of the claims. Embodiments of the present technology, together with further advantages, will be best understood by reference to the following detailed description taken in conjunction with the accompanying drawings.

### BRIEF DESCRIPTION OF THE DRAWINGS

FIG. 1 shows exemplary before and after photos of the results of using the topical compositions for preventing and treating scar formation of a surgical incision on a subject’s underarm according to some embodiments of the present technology.

FIG. 2 shows exemplary before and after photos of the results of using the topical compositions for preventing and treating scar formation of a surgical incision on a subject’s torso according to some embodiments of the present technology.

FIG. 3 shows exemplary before and after photos of the results of using the topical compositions for preventing and treating scar formation of a surgical incision on a subject’s facial area according to some embodiments of the present technology.

### DETAILED DESCRIPTION

Described herein are topical compositions for use in treating or preventing scars. According to the embodiments described herein, a set of topical compositions can be used in methods and protocols over the course of several months as discussed in detail below.

### Definitions

Unless defined otherwise, all technical and scientific terms used herein have the same meaning as commonly understood by those of ordinary skill in the art to which the present technology belongs. For the purposes of the present technology, the following terms are defined below.

The articles “a” and “an” are used herein to refer to one or to more than one (i.e., to at least one) of the grammatical object of the article. By way of example, “an element” means one element or more than one element. Likewise, any reference to singular includes plural embodiments, and any reference to more than one component may include a singular embodiment.

The term “about” means a quantity, level, value, number, frequency, percentage, dimension, size, amount, weight or length that varies by acceptable levels in the art. In some embodiments, such variation may be as much as 30, 25, 20, 15, 10, 9, 8, 7, 6, 5, 4, 3, 2 or 1% to a reference quantity, level, value, number, frequency, percentage, dimension, size, amount, weight or length. When the term “about” is used in conjunction with a numerical range, it modifies that range by extending the boundaries above and below the numerical values set forth.

As used herein, the terms “topical,” “apply,” “applying,” and “application,” refer to a mode of application as being directly to a part of a body surface (e.g., skin or mucous membrane). Unless specified otherwise, the administration can be in any amount or volume.

As used herein, the term “set” refers to a combination of two or more compositions (e.g., a cleanser and a treatment, a cleanser and two or more treatments, a treatment and one or more treatment).

As used herein, the terms “treatment,” and “treat” refer to the reduction or inhibition of the progression and/or formation of an ailment (e.g., scar or wound), the reduction or amelioration of the severity of the ailment, and/or the amelioration of one or more symptoms thereof resulting from the application of one or more topical compositions as described herein.

As used herein, the term “scar” refers to a mark on the skin or topical site, or a partially healed incision, wound, sore, burn, or injury site that has not yet healed completely, and where fibrous tissue has developed.

As used herein, the term “wound” refers to an opening of the topical site of the body (e.g., skin), where the external surface is not fully intact (e.g., sore, burn, cut, incision).

As used herein, the term “period of time” refers to any duration that is continuous or progressive. Unless otherwise specified, “period of time” can include any duration that is in the metric of seconds, minutes, hours, days, weeks, months, and years.

### Topical Compositions

In some embodiments, a set of topical compositions includes a cleansing composition and one or more treatment compositions. In certain embodiments, the set of topical compositions includes five treatment compositions: a first treatment composition, a second treatment composition, a third treatment composition, a fourth treatment composition, and a fifth treatment composition. In accordance with the embodiments described herein, any one of the compositions may be formulated to include one or more of the following primary ingredients or derivatives therefrom: aloe vera, persea (avocado), macadamia, tocopherol or vitamin E, lavender, allantoin, glycerin, purified snail mucin, oryza sativa, tocopherol, rosa gallica, salix alba, coconut oil, retinol, helianthus, and jojoba. The significance of each of these ingredients according to some embodiments is discussed below.

In some embodiments, the cleansing composition is formulated with ingredients that contain antibiotic properties to clean and soothe the scar or wound. In certain embodiments, the cleansing composition includes polydimethylsiloxane (silicone) and Rosmarinus officinalis (Rosemary) flower extract. In some embodiments, the silicone is present in an amount ranging from about 1% to about 10% by weight of the cleansing composition and the rosemary flower extract is present in an amount ranging from about 10% to about 20% by weight of the cleansing composition. In one embodiment, the silicone is present in an amount of about 5% by weight of the cleansing composition and the rosemary flower extract is present in an amount of about 15% by weight of the cleansing composition.

In some embodiments, the cleansing composition may include ingredients including, but not limited to, therapeutics, botanicals, and vehicles. In certain embodiments, the cleansing composition includes rosemary flower extract and silicone (as discussed above), as well as one or more of the following ingredients: decyl glucoside, Cocos nucifera (Coconut) oil, europaea (olive) fruit oil, Helianthus annuus (sunflower) seed oil, Aloe barbadenis (Aloe or aloe vera) leaf juice, citric acid, Butyrospermum parkii (shea) butter, and Rosmarinus officinalis (rosemary) oil. When included in the cleansing composition, the ingredient(s) may be present in the following amounts according to some embodiments:

| | |
|---|---|
| Decyl Glucoside | about 1-2% by weight |
| Cocos Nucifera (Coconut) Oil | about 15-25% by weight |
| Europaea (Olive) Fruit Oil | about 15-25% by weight |
| Helianthus Annuus (Sunflower) Seed Oil | about 10-20% by weight |
| Aloe Barbadenis (Aloe) Leaf Juice | about 5-15% by weight |
| Citric Acid | about 1-3% by weight |
| Butyrospermum Parkii (Shea) Butter | about 5-10% by weight |
| Rosmarinus Officinalis (Rosemary) Oil | about 1-5% by weight |

In one embodiment, the cleansing composition is formulated to include the following ingredients and in the amounts as indicated below:

| | |
|---|---|
| Decyl Glucoside | about 1.5% by weight |
| Cocos Nucifera (Coconut) Oil | about 20% by weight |
| Europaea (Olive) Fruit Oil | about 20% by weight |
| Helianthus Annuus (Sunflower) Seed Oil | about 15.5% by weight |
| Aloe Barbadenis (Aloe) Leaf Juice | about 10% by weight |
| Citric Acid | about 2% by weight |
| Silicone | about 5% by weight |
| Butyrospermum Parkii (Shea) Butter | about 8% by weight |
| Rosmarinus Officinalis (Rosemary) Oil | about 3% by weight |
| Rosemary Flower Extract | about 15% by weight |

In some embodiments, the first treatment composition is formulated with ingredients to treat inflammation and encourage the healing process of the scar or wound. In certain embodiments, the first treatment composition includes Aloe barbadensis (aloe or aloe vera) leaf juice, Macadamia ternifolia (macadamia) seed oil, and panthenol (vitamin B5). In some embodiments, the aloe leaf juice is present in an amount ranging from about 10% to about 20% by weight of the first treatment composition, the macadamia seed oil is present in an amount ranging from about 1% to about 3% by weight of the first treatment composition, and the vitamin B5 is present in an amount ranging from about 1% to about 3% by weight of the first treatment composition. In one embodiment, the aloe leaf juice is present in an amount of about 15% by weight of the first treatment composition, the macadamia seed oil is present in an amount of about 1.5% by weight of the first treatment composition, and the vitamin B5 is present in an amount of about 2% by weight of the first treatment composition.

In some embodiments, the first treatment composition may include ingredients including, but not limited to therapeutics, botanicals, and vehicles. In certain embodiments, the first treatment composition includes aloe leaf juice, macadamia seed oil, and vitamin B5 (as discussed above), as well as one or more of the following ingredients: Aloe barbadensis (aloe or aloe vera) leaf juice, aqua (deionized water), tocopheryl acetate (vitamin E), Helianthus annuus (sunflower) seed oil, glyceryl steswax, potassium stearate, Simmondsia chinensis (Jojoba) seed oil, Butyrospermum parkii (shea) butter, Calophyllum inophyllum (tamanu) seed oil, Macadamia ternifolia (macadamia) seed oil, Aleurites moluccana (kukui) seed oil, Persea gratissima (avocado) oil, Borago officinalis (borage) seed oil, Linum usitatissimum (flax) seed oil, Oenothera biennis (evening primrose) oil, Rosa canina (rosehip) fruit oil, panthenol (vitamin B5), Camellia sinensis (green tea) leaf extract, Morinda citrifolia (noni) extract, Passiflora incarnata (passionflower) extract, Calendula officinalis (calendula) extract, Chamomilla recutita (chamomile) extract, Lavandula angustifolia (lavender) extract, Arnica montana (arnica) extract, Centella asiatica (gotu kola) extract, Equisetum arvense (horsetail) extract, Geranium maculatum (wild geranium) extract, Taraxacum officinale (dandelion) extract, stearic acid, xanthan gum, sodium carbomer, and pentylene glycol. In certain embodiments, the first treatment composition includes aloe leaf juice, persea oil, macadamia nut seed oil, vitamin E, and lavender. When included in the first treatment composition, the ingredient(s) may be present in the following amounts according to some embodiments:

| | |
|---|---|
| Aqua (Deionized Water) | about 20-60% by weight |
| Tocopheryl Acetate (Vitamin E) | about 1-10% by weight |
| Helianthus Annuus (Sunflower) Seed Oil | about 1-5% by weight |
| Glyceryl Steswax | about 1-2% by weight |
| Potassium Stearate | about 1-2% by weight |
| Simmondsia Chinensis (Jojoba) Seed Oil | about 1-5% by weight |
| Butyrospermum Parkii (Shea) Butter | about 1-5% by weight |
| Calophyllum Inophyllum (Tamanu) Seed Oil | about 1-2% by weight |
| Aleurites Moluccana (Kukui) Seed Oil | about 1-2% by weight |
| Persea Gratissima (Avocado) Oil | about 1-3% by weight |
| Borago Officinalis (Borage) Seed Oil | about 1-2% by weight |
| Linum Usitatissimum (Flax) Seed Oil | about 0.5-2% by weight |
| Oenothera Biennis (Evening Primrose) Oil | about 0.5-2% by weight |
| Rosa Canina (Rosehip) Fruit Oil | about 0.5-2% by weight |
| Camellia Sinensis (Green Tea) Leaf Extract | about 1-3% by weight |
| Morinda Citrifolia (Noni) Extract | about 1-3% by weight |
| Passiflora Incarnata (Passionflower) Extract | about 0.5-2% by weight |
| Calendula Officinalis (Calendula) Extract | about 0.1-1% by weight |
| Chamomilla Recutita (Chamomile) Extract | about 0.5-2% by weight |
| Lavandula Angustifolia (Lavender) Extract | about 0.5-2% by weight |
| Arnica Montana (Arnica) Extract | about 0.5-2% by weight |
| Centella Asiatica (Gotu Kola) Extract | about 0.5-2% by weight |
| Equisetum Arvense (Horsetail) Extract | about 0.5-2% by weight |
| Geranium Maculatum (Wild Geranium) Extract | about 0.5-2% by weight |
| Taraxacum Officinale (Dandelion) Extract | about 0.5-2% by weight |
| Stearic Acid | about 1-2% by weight |
| Xanthan Gum | about 1-3% by weight |
| Sodium Carbomer | about 0.5-2% by weight |
| Pentylene Glycol | about 1-2% by weight |

In one embodiment, the first treatment composition is formulated to include the following ingredients and in the amounts as indicated below:

| | |
|---|---|
| Aloe Barbadensis (Aloe) Leaf Juice | about 15% by weight |
| Aqua (Deionized Water) | about 40% by weight |
| Tocopheryl Acetate (Vitamin E) | about 5% by weight |
| Helianthus Annuus (Sunflower) Seed Oil | about 3% by weight |
| Glyceryl Steswax | about 0.5% by weight |
| Potassium Stearate | about 0.3% by weight |
| Simmondsia Chinensis (Jojoba) Seed Oil | about 3% by weight |
| Butyrospermum Parkii (Shea) Butter | about 3% by weight |
| Calophyllum Inophyllum (Tamanu) Seed Oil | about 1.5% by weight |
| Macadamia Ternifolia (Macadamia) Seed Oil | about 1.5% by weight |
| Aleurites Moluccana (Kukui) Seed Oil | about 1.5% by weight |
| Persea Gratissima (Avocado) Oil | about 2% by weight |
| Borago Officinalis (Borage) Seed Oil | about 1.5% by weight |
| Linum Usitatissimum (Flax) Seed Oil | about 1% by weight |
| Oenothera Biennis (Evening Primrose) Oil | about 1% by weight |
| Rosa Canina (Rosehip) Fruit Oil | about 1% by weight |
| Panthenol (Vitamin B5) | about 2% by weight |
| Camellia Sinensis (Green Tea) Leaf Extract | about 2% by weight |
| Morinda Citrifolia (Noni) Extract | about 2% by weight |
| Passiflora Incarnata (Passionflower) Extract | about 1% by weight |
| Calendula Officinalis (Calendula) Extract | about 0.2% by weight |
| Chamomilla Recutita (Chamomile) Extract | about 1% by weight |
| Lavandula Angustifolia (Lavender) Extract | about 1% by weight |
| Arnica Montana (Arnica) Extract | about 1% by weight |
| Centella Asiatica (Gotu Kola) Extract | about 1% by weight |
| Equisetum Arvense (Horsetail) Extract | about 1% by weight |
| Geranium Maculatum (Wild Geranium) Extract | about 1% by weight |
| Taraxacum Officinale (Dandelion) Extract | about 1% by weight |
| Stearic Acid | about 0.5% by weight |
| Xanthan Gum | about 2% by weight |
| Sodium Carbomer | about 1% by weight |
| Pentylene Glycol | about 1.5% by weight |

In some embodiments, the second treatment composition is formulated with ingredients to moisturize and support the healing structures of the scar or wound. In certain embodiments, the second treatment composition includes Aloe barbadensis (aloe or aloe vera) leaf extract, sodium hyaluronate, and allantoin. In some embodiments, the aloe leaf extract is present in an amount ranging from about 1% to about 5% by weight of the second treatment composition, the sodium hyaluronate is present in an amount ranging from about 1% to about 5% by weight of the second treatment composition, and the allantoin is present in an amount ranging from about 1% to about 3% by weight of the second treatment composition. In one embodiment, the aloe leaf extract is present in an amount of about 3% by weight of the second treatment composition, the sodium hyaluronate is present in an amount of about 3% by weight of the second treatment composition, and the allantoin is present in an amount of about 2% by weight of the second treatment composition.

In some embodiments, the second treatment composition may include ingredients including, but not limited to therapeutics, botanicals, and vehicles. In certain embodiments, the second treatment composition includes aloe leaf extract, sodium hyaluronate, and allantoin (as discussed above), as well as one or more of the following ingredients: aqua (deionized water), glycerin, propylene glycol, ethylhexyl palmitate (palm oil derivate), sorbitan olivate, caprylic/capric triglyceride, paraffinum liquidum, squalane, dimethicone, isohexadecane, hydrogenated coco-glycerides (coconut oil), polysorbate 60, hydroxyethyl acrylate/sodium acryloyldimethyl taurate copolymer, sodium acrylate/sodium acryloyldimethyl taurate copolymer, Althaea officinalis (marsh-mallow) root extract, beta-glucan, ethylhexylglycerin. In certain embodiments, the second treatment composition includes aloe leaf extract, allantoin, and glycerin. When included in the second treatment composition, the ingredient(s) may be present in the following amounts according to some embodiments:

| | |
|---|---|
| Aqua (Deionized Water) | about 20-60% by weight |
| Glycerin | about 1-10% by weight |
| Propylene Glycol | about 0.1-1% by weight |
| Ethylhexyl Palmitate (Palm Oil Derivate) | about 0.01-1% by weight |
| Sorbitan Olivate | about 0.1-1% by weight |
| Caprylic/Capric Triglyceride | about 0.1-1 % by weight |
| Paraffinum Liquidum | about 1-10% by weight |
| Squalane | about 1-10% by weight |
| Dimethicone | about 0.5-2% by weight |
| Isohexadecane | about 1-10% by weight |
| Hydrogenated Coco-glycerides (Coconut Oil) | about 5-10% by weight |
| Polysorbate 60 | about 1-5% by weight |
| Hydroxyethyl Acrylate/Sodium Acryloyldimethyl Taurate Copolymer | about 1-3% by weight |
| Sodium Acrylate/Sodium Acryloyldimethyl Taurate Copolymer | about 1-3% by weight |
| Althaea Officinalis (Marsh-Mallow) Root Extract | about 1-10% by weight |
| Beta-Glucan | about 1-5% by weight |
| Ethylhexylglycerin | about 0.1-1% by weight |

In one embodiment, the second treatment composition is formulated to include the following ingredients and in the amounts as indicated below:

| | |
|---|---|
| Aqua (Deionized Water) | about 46% by weight |
| Glycerin | about 5% by weight |
| Propylene Glycol | about 0.4% by weight |
| Ethylhexyl Palmitate (Palm Oil Derivate) | about 0.1% by weight |
| Sorbitan Olivate | about 0.5% by weight |
| Caprylic/Capric Triglyceride | about 0.5% by weight |
| Paraffinum Liquidum | about 5% by weight |
| Squalane | about 5% by weight |
| Dimethicone | about 1% by weight |
| Isohexadecane | about 5% by weight |
| Hydrogenated Coco-glycerides (Coconut Oil) | about 8% by weight |
| Polysorbate 60 | about 3% by weight |
| Hydroxyethyl Acrylate/Sodium Acryloyldimethyl Taurate Copolymer | about 2% by weight |
| Sodium Acrylate/Sodium Acryloyldimethyl Taurate Copolymer | about 2% by weight |
| Althaea Officinalis (Marsh-Mallow) Root Extract | about 5% by weight |
| Beta-Glucan | about 3% by weight |
| Aloe Barbadensis (Aloe) Leaf Extract | about 3% by weight |
| Sodium Hyaluronate | about 3% by weight |
| Allantoin | about 2% by weight |
| Ethylhexylglycerin | about 0.5% by weight |

In some embodiments, the third treatment composition is formulated with ingredients to treat any pigmentation issues and reduce the appearance of the scar or wound. In certain embodiments, the third treatment composition includes purified snail mucin. In some embodiments, the purified snail mucin is present in an amount ranging from about 1% to about 10% by weight of the third treatment composition. In one embodiment, the purified snail mucin is present in an amount of about 5% by weight of the third treatment composition.

In some embodiments, the third treatment composition may include ingredients including, but not limited to therapeutics, botanicals, and vehicles. In certain embodiments, the third treatment composition includes purified snail mucin (as discussed above), as well as one or more of the following ingredients: aqua (Deionized Water), glycerol, Viola tricolor (wild pansy) extract, Aloe barbadensis (aloe or aloe vera) leaf juice, Butyrospermum parkii (shea tree), Oryza sativa (rice) germ, Rosa gallica (French rose) extract, caprylic / capric triglyceride, Squalane, Betaine, Dimethicone, Xanthan Gum, tocopherol, laurocapram, and sodium hyaluronate. In certain embodiments, the third treatment composition includes purified snail mucin, oryza sativa, and rosa gallica. When included in the third treatment composition, the ingredient(s) may be present in the following amounts according to some embodiments:

| | |
|---|---|
| Aqua (Deionized Water) | about 20-60% by weight |
| Glycerol | about 1-10% by weight |
| Snail Mucin Extract | about 1-10% by weight |
| Viola Tricolor (Wild Pansy) Extract | about 1-5% by weight |
| Aloe Barbadensis (Aloe) Leaf Juice | about 5-10% by weight |
| Butyrospermum Parkii (Shea Tree) | about 1-10%by weight |
| Oryza Sativa (Rice) Germ | about 0.1-1% by weight |
| Rosa Gallica (French Rose) Extract | about 1-3% by weight |
| Caprylic / Capric Triglyceride | about 5-10% by weight |
| Squalane | about 0.5-2% by weight |
| Betaine | about 1-10% by weight |
| Dimethicone | about 1-5% by weight |
| Xanthan Gum | about 1-5% by weight |
| Tocopherol (Vitamin E) | about 1-10%by weight |
| Laurocapram | about 1-2% by weight |
| Sodium Hyaluronate | about 0.1-1% by weight |

In one embodiment, the third treatment composition is formulated to include the following ingredients and in the amounts as indicated below:

| | |
|---|---|
| Aqua (Deionized Water) | about 45% by weight |
| Glycerol | about 5% by weight |
| Snail Mucin Extract | about 5% by weight |
| Viola Tricolor (Wild Pansy) Extract | about 3% by weight |
| Aloe Barbadensis (Aloe) Leaf Juice | about 8% by weight |
| Butyrospermum Parkii (Shea Tree) | about 4.5% by weight |
| Oryza Sativa (Rice) Germ | about 0.5% by weight |
| Rosa Gallica (French Rose) Extract | about 2% by weight |
| Caprylic / Capric Triglyceride | about 8% by weight |
| Squalane | about 1% by weight |
| Betaine | about 5% by weight |
| Dimethicone | about 3% by weight |
| Xanthan Gum | about 3% by weight |
| Tocopherol (Vitamin E) | about 5% by weight |
| Laurocapram | about 1.5% by weight |
| Sodium Hyaluronate | about 0.5% by weight |

In some embodiments, the fourth treatment composition is formulated with ingredients to decrease the appearance of the scar or wound. In certain embodiments, the fourth treatment composition includes tocopherol. In some embodiments, the tocopherol is present in an amount ranging from about 1% to about 9% by weight of the fourth treatment composition. In one embodiment, the tocopherol is present in an amount of about 5% by weight of the fourth treatment composition.

In some embodiments, the fourth treatment composition may include ingredients including, but not limited to, therapeutics, botanicals, and vehicles. In certain embodiments, the fourth treatment composition includes tocopherol (as discussed above), as well as one or more of the following ingredients: aqua (deionized water), Theobroma cacao (cocoa) butter, Butyrospermum parkii (shea) butter, Cocos nucifera (coconut) oil, glycerin, polydimethylsiloxane (silicone), xanthan gum, Salix alba (white willow) bark extract, Rosmarinus officianalis (rosemary) leaf extract, and Azadirachta indica (neem) seed oil. In certain embodiments, the fourth treatment composition includes tocopherol, salix alba, and coconut oil. When included in the fourth treatment composition, the ingredient(s) may be present in the following amounts according to some embodiments:

| | |
|---|---|
| Aqua (Deionized Water) | about 40-60% by weight |
| Theobroma Cacao (Cocoa) Butter | about 10-20% by weight |
| Butyrospermum Parkii (Shea) Butter | about 1-10% by weight |
| Cocos Nucifera (Coconut) Oil | about 5-10% by weight |
| Glycerin | about 1-10% by weight |
| Polydimethylsiloxane (Silicone) | about 0.1-1% by weight |
| Xanthan Gum | about 1-10% by weight |
| Salix Alba (White Willow) Bark Extract | about 1-5% by weight |
| Rosmarinus Officianalis (Rosemary) Leaf Extract | about 1-5% by weight |
| Azadirachta Indica (Neem) Seed Oil | about 1-3% by weight |

In one embodiment, the fourth treatment composition is formulated to include the following ingredients and in the amounts as indicated below:

| | |
|---|---|
| Aqua (Deionized Water) | about 48.5% by weight |
| Theobroma Cacao (Cocoa) Butter | about 15% by weight |
| Butyrospermum Parkii (Shea) Butter | about 5% by weight |
| Cocos Nucifera (Coconut) Oil | about 8% by weight |
| Glycerin | about 5% by weight |
| Tocopherol (Vitamin E) | about 5% by weight |
| Polydimethylsiloxane (Silicone) | about 0.5% by weight |
| Xanthan Gum | about 5% by weight |
| Salix Alba (White Willow) Bark Extract | about 3% by weight |
| Rosmarinus Officianalis (Rosemary) Leaf Extract | about 3% by weight |
| Azadirachta Indica (Neem) Seed Oil | about 2% by weight |

In some embodiments, the fifth treatment composition is formulated with ingredients to fade and smooth the scar or wound. In certain embodiments, the fifth treatment composition includes retinol or a derivative thereof. In some embodiments, the retinol is present in an amount ranging from about 1% to about 5% by weight of the fifth treatment composition. In one embodiment, the retinol is present in an amount of about 2.5% by weight of the fifth treatment composition.

In some embodiments, the fifth treatment composition may include ingredients including, but not limited to, therapeutics, botanicals, and vehicles. In certain embodiments, the fifth treatment composition includes retinol or a derivative thereof (as discussed above), as well as one or more of the following ingredients: aqua (deionized Water), Aloe barbadensis (aloe or aloe vera) extract, Helianthus annuus (sunflower) oil, glyceryl stearate, stearic acid, Cassia angustifolia (senna) seed, vegetable glycerin, Simmondsia chinensis (jojoba) oil, Camellia sinensis (green tea) leaf extract, polydimethylsiloxane (silicone), Resina propoli (beeswax acid), Butyrospermum parkii (shea) butter, panthenol (vitamin B5), tocopherol (vitamin E), xanthan gum, phospholipids (sunflower lecithin). pentylene glycol, polysorbate 20, potassium sorbate, and ethylhexyl glycerin. In certain embodiments, the fifth treatment composition includes retinol, helianthus, and jojoba. When included in the fifth treatment composition, the ingredient(s) may be present in the following amounts according to some embodiments:

| | |
|---|---|
| Aqua (Deionized Water) | about 40-60% by weight |
| Aloe Barbadensis (Aloe) Extract | about 5-10% by weight |
| Helianthus Annuus (Sunflower) Oil | about 1-10% by weight |
| Glyceryl Stearate | about 0.1-1% by weight |
| Stearic Acid | about 5-10% by weight |
| Cassia Angustifolia (Senna) Seed | about 1-5% by weight |
| Vegetable Glycerin | about 1-3% by weight |
| Simmondsia Chinensis (Jojoba) Oil | about 1-10% by weight |
| Camellia Sinensis (Green Tea) Leaf Extract | about 1-10% by weight |
| Polydimethylsiloxane (Silicone) | about 0.1-1% by weight |
| Resina Propoli (Beeswax Acid) | about 0.1-1% by weight |
| Butyrospermum Parkii (Shea) Butter | about 1-10% by weight |
| Panthenol (Vitamin B5) | about 1-3% by weight |
| Tocopherol (Vitamin E) | about 1-3% by weight |
| Xanthan Gum | about 1-5% by weight |
| Phospholipids (Sunflower Lecithin) | about 0.1-1% by weight |
| Pentylene Glycol | about 0.1-1% by weight |
| Polysorbate 20 | about 0.1-1% by weight |
| Potassium Sorbate | about 0.1-1% by weight |
| Ethylhexyl Glycerin | about 0.1-1% by weight |

In one embodiment, the fifth treatment composition is formulated to include the following ingredients and in the amounts as indicated below:

| | |
|---|---|
| Aqua (Deionized Water) | about 45.5% by weight |
| Aloe Barbadensis (Aloe) Extract | about 8% by weight |
| Helianthus Annuus (Sunflower) Oil | about 5% by weight |
| Glyceryl Stearate | about 0.5% by weight |
| Stearic Acid | about 8% by weight |
| Cassia Angustifolia (Senna) Seed | about 3% by weight |
| Vegetable Glycerin | about 2% by weight |
| Retinol 2.5% | about 2.5% by weight |
| Simmondsia Chinensis (Jojoba) Oil | about 5% by weight |
| Camellia Sinensis (Green Tea) Leaf Extract | about 5% by weight |
| Polydimethylsiloxane (Silicone) | about 0.5% by weight |
| Resina Propoli (Beeswax Acid) | about 0.5% by weight |
| Butyrospermum Parkii (Shea) Butter | about 5% by weight |
| Panthenol (Vitamin B5) | about 2% by weight |
| Tocopherol (Vitamin E) | about 2% by weight |
| Xanthan Gum | about 3% by weight |
| Phospholipids (Sunflower Lecithin) | about 0.5% by weight |
| Pentylene Glycol | about 0.5% by weight |
| Polysorbate 20 | about 0.5% by weight |
| Potassium Sorbate | about 0.5% by weight |
| Ethylhexyl Glycerin | about 0.5% by weight |

In certain embodiments, the cleansing and treatment compositions described herein may be formulated as a solution, suspension, cream, ointment, lotion, serum, gel, fluid/liquid, paste, transdermal patch, foam, spray, powder, liquid powder, cream powder, oil, butter, elixir, concentrate, solid, semi-solid, or any other form suitable for topical application.

In some embodiments, the set of topical compositions is a cleansing composition and one treatment compositions, wherein the set contains the ingredients Aloe barbadensis (aloe or aloe vera) leaf juice, snail mucin extract, tocopherol, and retinol. In some embodiments, the set of topical compositions is a cleansing composition, a first treatment composition, and a second treatment composition, wherein the set contains the ingredients Aloe barbadensis (aloe or aloe vera) leaf juice, snail mucin extract, tocopherol, and retinol. In some embodiments, the set of topical compositions is a cleansing composition, a first treatment composition, a second treatment composition, and a third treatment composition, wherein the set contains the ingredients Aloe barbadensis (aloe or aloe vera) leaf juice, snail mucin extract, tocopherol, and retinol. In some embodiments, the set of topical compositions is a cleansing composition, a first treatment composition, a second treatment composition, a third treatment composition, and a fourth treatment composition, wherein the set contains the ingredients Aloe barbadensis (aloe or aloe vera) leaf juice, snail mucin extract, tocopherol, and retinol. In some embodiments, the set of topical compositions is a cleansing composition, a first treatment composition, a second treatment composition, a third treatment composition, a fourth treatment composition, and a fifth treatment composition, wherein the set contains the ingredients Aloe barbadensis (aloe or aloe vera) leaf juice, snail mucin extract, tocopherol, and retinol.

### Kits

While the set of topical compositions described in the embodiments above may be provided individually and/or independent from each other, the full set may alternatively be provided in a kit so the set of topical compositions can be sold together.

In some embodiments, the kits include a container that holds (i) a set of two containers that contain the cleansing composition, the first treatment composition, and (ii) an instructional insert card. The container holding the contents of the kit may be any suitable container, e.g., a box.

In some embodiments, the kits include a container that holds (i) a set of three containers that contain the cleansing composition, the first treatment composition, the second treatment composition, and (ii) an instructional insert card. The container holding the contents of the kit may be any suitable container, e.g., a box.

In some embodiments, the kits include a container that holds (i) a set of four containers that contain the cleansing composition, the first treatment composition, the second treatment composition, the third treatment composition, and (ii) an instructional insert card. The container holding the contents of the kit may be any suitable container, e.g., a box.

In some embodiments, the kits include a container that holds (i) a set of five containers that contain the cleansing composition, the first treatment composition, the second treatment composition, the third treatment composition, the fourth treatment composition, and (ii) an instructional insert card. The container holding the contents of the kit may be any suitable container, e.g., a box.

In some embodiments, the kits include a container that holds (i) a set of six containers that contain the cleansing composition, the first treatment composition, the second treatment composition, the third treatment composition, the fourth treatment composition, and the fifth treatment composition, and (ii) an instructional insert card. The container holding the contents of the kit may be any suitable container, e.g., a box.

In some embodiments, the containers to contain the cleansing composition and the treatment compositions are jars, tubes, bottles, dispensers, pumps, sprayers, or any other suitable cosmetic packaging container in any suitable material (e.g., glass, metal, plastic, etc.) for the formulations used. In one embodiment, the cleansing composition is contained in a tube, and the treatment compositions are contained in jars or bottles. In other embodiments, the cleansing composition is contained in a tube and the treatment compositions are impregnated in bandages (e.g., gauze, non-woven sponge, ropes, strip, towelette, etc.). In such embodiments, the kit includes a sufficient number of impregnated bandages to complete the treatment.

### Methods and Protocols for Treating or Preventing Scars

The topical compositions are used in methods for treating or preventing a scar according to the embodiments discussed herein. In some embodiments, the topical compositions are applied to a wound or scar by a subject having received the wound or scar. Any wound with the potential to form a scar (or has formed an existing scar) can be treated using the methods described herein including, but not limited to, a surgical incision, a traumatic laceration, a burn, a sore, or wounds from radiation damage or lasers.

According to some embodiments, the methods and protocols are started within a week of receiving the wound to optimize the treatment. In other embodiments, the methods and protocols described herein may be started within 6 months of receiving the wound, i.e., when the scar is less than 6 months old. Although optimal treatment should begin within 6 months of receiving the wound, use of the methods and protocols described herein after a scar is 6 months old would likely result in improvement of the scar.

In some embodiments, the methods of treatment include a first step of applying a cleansing composition in combination with a first treatment composition to the wound or scar. The cleansing composition and first treatment composition may be any of such compositions described herein according to some embodiments. In accordance with such embodiments, the cleansing composition is applied first to clean the wound or scar, followed by applying the first treatment composition to the cleaned wound or scar. The first step is continued for a first period of time. In certain embodiments, the first step is continued for a period of between about 2 weeks to about 6 weeks. In one embodiment, the first step is continued for a period of about one month. In some embodiments, the cleansing composition and the first treatment composition are applied at least once daily for the duration of the first period of time. In other embodiments, the cleansing composition and the first treatment composition are applied twice daily for the duration of the first period of time. In yet other embodiments, the cleansing composition and the first treatment composition are applied more than twice daily for the duration of the first period of time.

In some embodiments, the methods of treatment include a second step of applying a second treatment composition to the wound or scar. The second treatment composition may be any of such compositions described herein according to some embodiments. The second step is continued for a second period of time, and in some embodiments, is started once the first period of time has elapsed, i.e., the second step is performed after the first period of time has ended. In certain embodiments, the second step is continued for a period of between about 6 weeks to about 14 weeks. In one embodiment, the second step is continued for a period of about two or three months. In some embodiments, the second treatment composition is applied at least once daily for the duration of the second period of time. In other embodiments, the second treatment composition is applied twice daily for the duration of the second period of time. In yet other embodiments, the second treatment composition is applied more than twice daily for the duration of the second period of time.

In some embodiments, the methods of treatment include a third step of applying a third treatment composition to the wound or scar. The third treatment composition may be any of such compositions described herein according to some embodiments. The third step is continued for a third period of time, and in some embodiments, is started once the second period of time has elapsed, i.e., the third step is performed after the second period of time has ended. In certain embodiments, the third step is continued for a period of between about 6 weeks to about 14 weeks. In one embodiment, the third step is continued for a period of about two or three months. In some embodiments, the third treatment composition is applied at least once daily for the duration of the third period of time. In other embodiments, the third treatment composition is applied twice daily for the duration of the third period of time. In yet other embodiments, the third treatment composition is applied more than twice daily for the duration of the third period of time.

In some embodiments, the methods of treatment include a fourth step of applying a fourth treatment composition to the wound or scar. The fourth treatment composition may be any of such compositions described herein according to some embodiments. The fourth step is continued for a fourth period of time, and in some embodiments, is started once the third period of time has elapsed, i.e., the fourth step is performed after the third period of time has ended. In certain embodiments, the fourth step is continued for a period of between about 10 weeks to about 18 weeks. In one embodiment, the fourth step is continued for a period of about three or four months. In some embodiments, the fourth treatment composition is applied at least once daily for the duration of the fourth period of time. In other embodiments, the fourth treatment composition is applied twice daily for the duration of the fourth period of time. In yet other embodiments, the fourth treatment composition is applied more than twice daily for the duration of the fourth period of time.

In some embodiments, the methods of treatment include a fifth step of applying a fifth treatment composition to the wound or scar. The fifth treatment composition may be any of such compositions described herein according to some embodiments. The fifth step is continued for a fifth period of time, and in some embodiments, is started once the fourth period of time has elapsed, i.e., the fifth step is performed after the fourth period of time has ended. In certain embodiments, the fifth step is continued for a period of between about 6 weeks to about 14 weeks. In one embodiment, the fifth step is continued for a period of about two or three months. In some embodiments, the fifth treatment composition is applied at least once daily for the duration of the fifth period of time. In other embodiments, the fifth treatment composition is applied twice daily for the duration of the fifth period of time. In yet other embodiments, the fifth treatment composition is applied more than twice daily for the duration of the fifth period of time.

According to some embodiments, the first, second, third, fourth, and fifth steps may be performed sequentially in their entirety. Alternatively, one or more of the first, second, third, fourth, and fifth steps may be performed in different orders, and/or one or more of the first, second, third, fourth, and fifth steps may be omitted.

Treatment of wounds and scars according to some embodiments described herein include a scar treatment protocol that includes several phases over the course of about a year. In some embodiments, the scar treatment protocol includes a first phase (or Phase I), which includes a cleanser composition and a first treatment composition that are applied to a wound or scar at least once daily for about a month. In such embodiments, the cleanser composition is applied in conjunction with the first treatment composition, where the cleanser is applied in advance of the first treatment composition. The cleansing composition and first treatment composition may be any of such compositions described herein. In some embodiments, the cleanser composition and the first treatment composition are applied twice daily or more than twice daily.

In some embodiments, the scar treatment protocol includes a second phase (or Phase II), which includes a second treatment composition that is applied to a wound or scar at least once daily for about two or three months. The second treatment composition may be any of such compositions described herein. In some embodiments, the second treatment composition is applied twice daily or more than twice daily.

In some embodiments, the scar treatment protocol includes a third phase (or Phase III), which includes a third treatment composition that is applied to a wound or scar at least once daily for about two or three months. The second treatment composition may be any of such compositions described herein. In some embodiments, the second treatment composition is applied twice daily or more than twice daily.

In some embodiments, the scar treatment protocol includes a fourth phase (or Phase IV), which includes a fourth treatment composition that is applied to a wound or scar at least once daily for about three or four months. The second treatment composition may be any of such compositions described herein. In some embodiments, the second treatment composition are applied twice daily or more than twice daily.

In some embodiments, the scar treatment protocol includes a fifth phase (or Phase V), which includes a fifth treatment composition that is applied to a wound or scar at least once daily for about two or three months. The fifth treatment composition may be any of such compositions described herein according to some embodiments. In some embodiments, the fifth treatment composition is applied twice daily or more than twice daily.

In one embodiment, the scar treatment protocol includes the following instructions:

| | | | |
|---|---|---|---|
| Phase | Timeline | Duration | Instructions |
| Phase I Cleanser | Month 0-1 | 1 month | Apply a dime-sized amount to the scar. Rinse thoroughly. Use twice daily. |
| Phase I Ointment | Month 0-1 | 1 month | Apply a moderate coating of ointment covering the entire scar twice daily. |
| Phase II Ointment | Months 1-3 | 2 months | Apply a moderate coating of ointment covering the entire scar twice daily. |
| Phase III Ointment | Months 3-5 | 2 months | Apply a moderate coating of ointment covering the entire scar twice daily. |
| Phase IV Ointment | Months 5-9 | 4 months | Apply a moderate coating of ointment covering the entire scar twice daily. |
| Phase V Ointment | Months 9-12 | 3 months | Apply a moderate coating of ointment covering the entire scar twice daily. |

According to some embodiments, the first, second, third, fourth, and fifth phases may occur sequentially in their entirety. Alternatively, one or more of the first, second, third, fourth, and fifth phases may occur in different orders, and/or one or more of the first, second, third, fourth, and fifth steps may be omitted.

The treatment compositions used in the methods and protocols described herein may be topically applied directly to a scar or a wound by a user in accordance with some embodiments. In other embodiments, a bandage (e.g., gauze, non-woven sponge, ropes, strip, towelette, etc.) may be impregnated or saturated with a treatment composition, and the impregnated bandage may be used to dress the wound or scar to topically apply the treatment composition.

From the foregoing, it will be appreciated that specific embodiments of the invention have been described herein for purposes of illustration, but that various modifications may be made without deviating from the scope of the invention. Accordingly, the invention is not limited except as by the appended claims.

### EXAMPLES

### Example 1: A System for Healing and Preventing Scars After Surgery

The scar protocol and system described below is an effective way to prevent scarring. Developed to treat healing wounds or scars for the first year following a surgical procedure, the scar protocol is not limited to treatment after a surgery and can also improve the look and feel of all scars. Formulated by a plastic surgeon, Scar Protocol works well for treating tummy tuck scars, breast lift scars, and facelift scars, but can also treat and prevent other surgical scars (e.g., C-section scars) and scars that occur due to injuries (e.g., laceration scars, scars from stitches). It is safe to use on any external surface of the body.

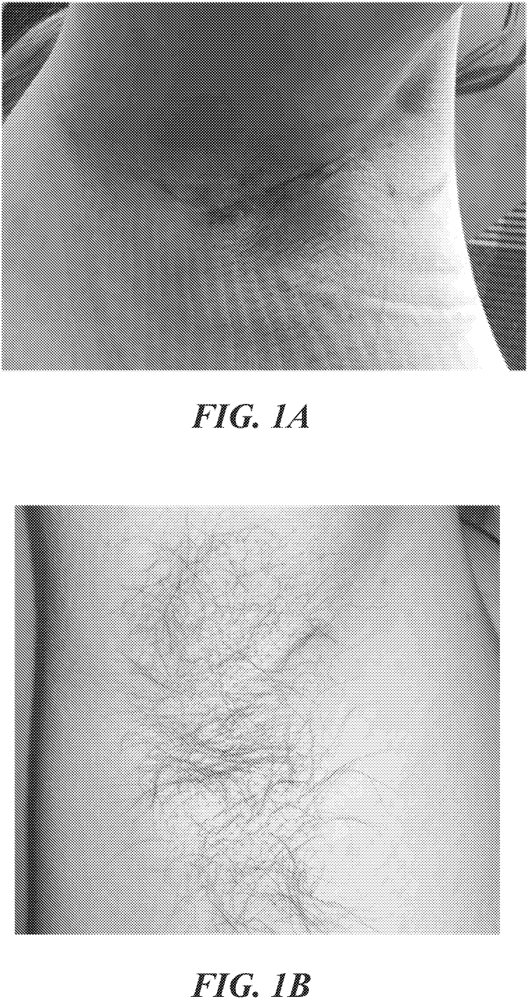

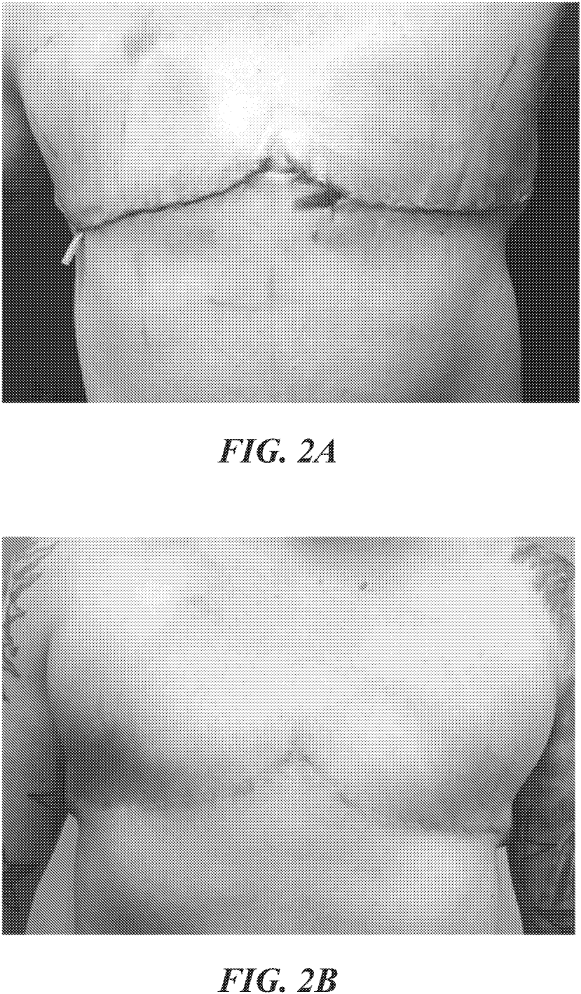

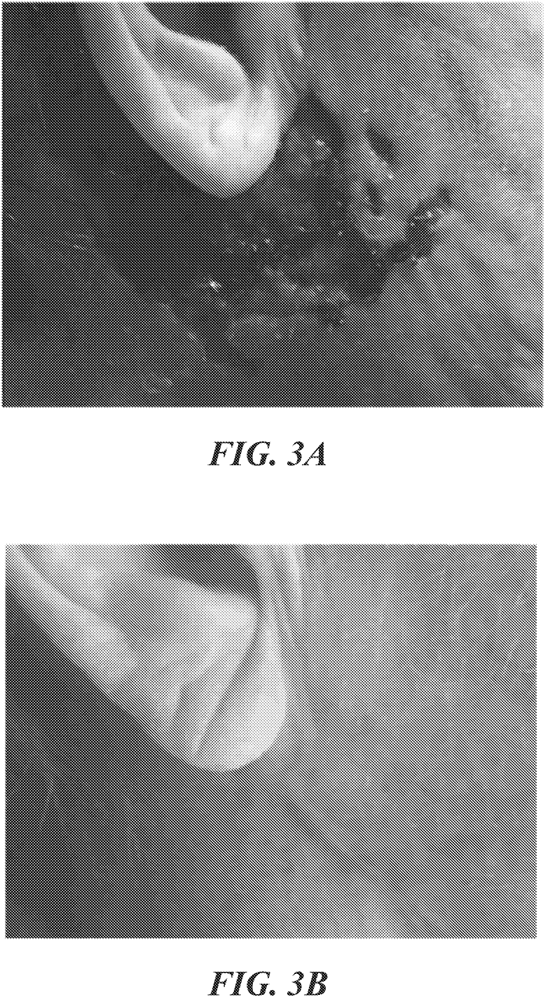

Used over the course of a year, the scar protocol along with the scar creams used in the protocol results in a comprehensive scar treatment system with proven results as shown, for example, in the before and after photos in FIGS. 1-3. Each phase of scar treatment protocol corresponds to a particular stage of healing. For that reason, the scar protocol is most effective when started soon after receiving the wound or scar. However, the ingredients contain multiple properties meant to smooth, fade, and prevent noticeable scarring, making the system effective even after the scar has formed or started to form. Thus, while the scar protocol works optimally for fresh scars (6 months or newer), it can also provide benefits for older scars.

Scar Protocol is safe for all skin types and tones. It contains soothing ingredients to moisturize and nourish the skin while resurfacing the scar. The scar creams used in the protocol are provided in a kit. The contents of which are sufficient to treat or prevent a scar for the entire length of the treatment protocol, which is about 12 months. Each kit includes one (1) tube of natural cleanser; five (5) jars of clinically tested natural treatment creams(Phase I, Phase II, Phase III, Phase IV, and Phase V); and one (1) instructional insert card that explains how to use the creams and cleanser. The treatment creams and the tube of cleanser are packaged in a reinforced velvet box.

All of the ingredients--with the exception of silicone--come from natural sources. Some of the notable ingredients of compositions include purified snail mucus, retinol, Vitamin E, and aloe vera. Instructions for use and formulations for each phase is discussed in detail below.

### Phase I

Treatment for prevention or minimizing a scar begins with Phase I, which includes a cleanser and a first scar treatment cream. Phase I is started one week after obtaining the wound or scar (e.g., one week after surgery) and lasts for the first month of the full 12 month protocol. It is best to start the scar system within six months of receiving the wound or scar for effective and optimal results. The ingredients of Phase I focus on giving the healing process a healthy start.

The cleanser and scar treatment cream are used concurrently. This helps to clean the surgical incision area, as well as helps keep it moisturized, nourished and provide important protection from free radicals and the harsh environment. Combining the cleanser with the specially formulated scar treatment cream makes Phase I superior to other scar systems and is important to its effectiveness.

Since Phase I encompasses the period soon after receiving the wound or scar, the formulation is designed to focus on promoting healthy healing of the wound and keeping the skin moisturized. The ingredients of Phase I are formulated to treat the freshly forming scar with ingredients to soothe the skin and help prevent infection. The cleanser and first treatment cream are formulated to include the ingredients shown in Tables 1 and 2 below.

TABLE 1

Cleanser Ingredients (by total weight of composition)

| Ingredient | % by weight |
|---|---|
| Decyl Glucoside | 1.5 |
| Cocos Nucifera (Coconut) Oil | 20 |
| Europaea (Olive) Fruit Oil | 20 |
| Helianthus Annuus (Sunflower) Seed Oil | 15.5 |
| Aloe Barbadenis (Aloe) Leaf Juice | 10 |
| Citric Acid | 2 |
| Polydimethylsiloxane (Silicone) | 5 |
| Butyrospermum Parkii (Shea) Butter | 8 |
| Rosmarinus Officinalis (Rosemary) Oil | 3 |
| Rosmarinus Officinalis (Rosemary) Flower Extract | 15 |
| TOTAL | 100 |

TABLE 2

First Treatment Cream Ingredients (by total weight of composition)

| Ingredient | % by weight |
|---|---|
| Aloe Barbadensis (Aloe) Leaf Juice | 15 |
| Aqua (Deionized Water) | 40 |
| Tocopheryl Acetate (Vitamin E) | 5 |
| Helianthus Annuus (Sunflower) Seed Oil | 3 |
| Glyceryl Steswax | 0.5 |
| Potassium Stearate | 0.3 |
| Simmondsia Chinensis (Jojoba) Seed Oil | 3 |
| Butyrospermum Parkii (Shea) Butter | 3 |
| Calophyllum Inophyllum (Tamanu) Seed Oil | 1.5 |
| Macadamia Ternifolia (Macadamia) Seed Oil | 1.5 |
| Aleurites Moluccana (Kukui) Seed Oil | 1.5 |
| Persea Gratissima (Avocado) Oil | 2 |
| Borago Officinalis (Borage) Seed Oil | 1.5 |
| Linum Usitatissimum (Flax) Seed Oil | 1 |
| Oenothera Biennis (Evening Primrose) Oil | 1 |
| Rosa Canina (Rosehip) Fruit Oil | 1 |
| Panthenol (Vitamin B5) | 2 |
| Camellia Sinensis (Green Tea) Leaf Extract | 2 |
| Morinda Citrifolia (Noni) Extract | 2 |
| Passiflora Incarnata (Passionflower) Extract | 1 |
| Calendula Officinalis (Calendula) Extract | 0.2 |
| Chamomilla Recutita (Chamomile) Extract | 1 |
| Lavandula Angustifolia (Lavender) Extract | 1 |
| Arnica Montana (Arnica) Extract | 1 |
| Centella Asiatica (Gotu Kola) Extract | 1 |
| Equisetum Arvense (Horsetail) Extract | 1 |
| Geranium Maculatum (Wild Geranium) Extract | 1 |
| Taraxacum Officinale (Dandelion) Extract | 1 |
| Stearic Acid | 0.5 |
| Xanthan Gum | 2 |
| Sodium Carbomer | 1 |
| Pentylene Glycol | 1.5 |
| TOTAL | 100 |

Phase I is started at the beginning of the healing process. During this stage, basic care is required to prevent infection and ensure that the subject’s body does not overcorrect and lead to a highly visible scar.

Phase I is the only step containing two separate products meant to work together directly. First, the cleanser keeps the scar clean with ingredients that contain natural antibiotic properties. The cleanser is used twice a day to clean and soothe the wound or scar. After using the cleanser, the Phase I (i.e., first) scar treatment cream is applied twice daily, to fully cover the scar. The first scar treatment cream treats inflammation and encourages the healing process with its combination of ingredients.

Phase I is important because the subject’s body reacts to wounds within seconds to increase the blood supply to the area and to begin producing collagen to create a new layer of skin over the wound. This new tissue is known as scar tissue and has a distinct look and feel from intact skin. Scarring does not appear until the wound has healed. The scar protocol described herein works to prevent scarring before it even forms. For example, Phase I of the scar protocol increases the quality of the new tissue forming over the scar tissue. In the long run, this can decrease the pigment of the scar as well as smooth the texture.

Phase I includes a healthy dose of aloe vera and vitamin E to moisturize and cleanse the wound or scar. This combination of ingredients helps promote healing, initial epithelialization and minimizes inflammation. Phase I’s formulation offers anti-inflammatory properties and antioxidants for healthy healing. Other notable ingredients of Phase I include Persea (avocado), macadamia, and lavender. These Phase I ingredients have several properties that contribute to the efficacy of the treatment cream and thus, the protocol.

Aloe Vera. Aloe vera comes from a succulent grown in tropical and semi-tropical environments. Aloe vera products are commonly used to treat a sunburn or irritated dry skin during the winter months. That is because aloe vera contains complex carbohydrates, vitamin C, and other antioxidants. It can provide some pain relief and treat all forms of irritated skin; from dry skin to burn treatments. Combination of aloe vera with the other ingredients provides significant advantages to a subject’s skin and surgical incision.

Persea. The oil of Persea (i.e., avocado) has many skin benefits. Persea Gratissima, or avocado oil, is rich in fatty acids, vitamins, and minerals. It can act as a natural sunscreen, which is important because the sun can damage the skin quickly, make scarring worse, and hinder the healing of scars. Avocado oil can also boost collagen production that is important to the production of new skin and healing an incision after surgery. Along with helping along the healing process, avocado oil is used in the treatment of chapped itchy skin to relieve discomfort and support natural moisture. Thus, when used in the scar protocol described herein, avocado oil works as a way to protect the surgical site or other wound from the sun and elements, and to provide moisture and relief from itchiness. This can prevent infection of the incision or wound and keeps the surrounding skin healthy so that it can work to optimally heal the surgical site without forming an unsightly visible scar.

Macadamia. Like avocado oil, macadamia seed oil, contains many fatty acids and antioxidants. Especially notable is that macadamia oil contains palmitoleic acid. Palmitoleic acid is an antioxidant that speeds up healing and contributes to healthy cell membranes and regeneration. This can support the healing process and ensure that the current and new skin cells are healthy. Another remarkable benefit of macadamia oil is that it controls sebum production. Sebum is the oil in our skin that-when it builds up-can cause acne problems and lead to oily skin. Macadamia oil controls the amount of sebum on skin and how much is produced. This prevents clogging of pores and the additional buildup of oils near the wound or scar. Macadamia oil also contains phytosterols which contain anti-inflammatory benefits which will aid in recovery and make the healing process more comfortable.

Vitamin E. Vitamin E is found in many nut and fruit oils. Vitamin E protects against free radicals and environmental pollution to the skin and wound site. This is critical to preventing infection and nourishing the skin for optimal healing of the surgical site or other wound. Vitamin E provides several general skin benefits that are also beneficial in wound healing. For example, vitamin E supplements SPF and sun protection, helps reduce and correct sun damage, and helps keep the skin hydrated. In the scar treatment protocol, vitamin E also prevents scars from forming and healing perfectly, which reduces the appearance of visible scar tissue on the skin and the initial formation of scarring.

Lavender. Most people are probably more familiar with the scent of lavender versus the skin benefits it can yield. Lavender can boost anti-inflammatory properties (like those found in macadamia oil) and can kill bacteria that enters pores. Lavender oil can also be used to prevent acne because it prevents harmful pollutants and microbes from penetrating into the skin. It can also reduce redness at the wound site. As part of Phase I, lavender oil is important for protecting the healing incision. And, like other fruit and plant based oils, lavender oil contributes to moisturized skin.

### Phase II

The scar treatment protocol continues with Phase II, which includes a second scar treatment cream. Phase II begins after Phase I is completed, starting at the beginning of month two of the 12 month protocol, and lasting for about two months (or until the cream runs out). The second scar treatment cream is a moisturizing cream that includes ingredients to support the healing structures of the scar. Applying Phase II follows the same instructions as the Phase I cream (twice daily), ensuring that the cream fully covers the scar.

At the time Phase II is started, the scar should be somewhere between the first two stages of healing. Consequently, the subject may still experience some inflammation and pain even though the wound has visibly begun to heal. It is at this point when scar formation begins, which makes Phase II’s focus on important moisture. During this stage of scarring, new skin and tissues are forming and the subject may notice some new tissue formation. Keeping the surrounding skin healthy and moisturized will support these new structures. Thus, the second treatment cream provides an environment to support long lasting moisture and to encourage the skin to heal with minimal scarring, and the subject should see the incision or wounded area start to lose its redness and heal throughout months two and three. The second treatment cream is formulated to include the ingredients shown in Table 3 below.

TABLE 3

Second Treatment Cream Ingredients (by total weight of composition)

| Ingredient | % by weight |
|---|---|
| Aqua (Deionized Water) | 46 |
| Glycerin | 5 |
| Propylene Glycol | 0.4 |
| Ethylhexyl Palmitate (Palm Oil Derivate) | 0.1 |
| Sorbitan Olivate | 0.5 |
| Caprylic/Capric Triglyceride | 0.5 |
| Paraffinum Liquidum | 5 |
| Squalane | 5 |
| Dimethicone | 1 |
| Isohexadecane | 5 |
| Hydrogenated Coco-glycerides (Coconut Oil) | 8 |
| Polysorbate 60 | 3 |
| Hydroxyethyl Acrylate/Sodium Acryloyldimethyl Taurate Copolymer | 2 |
| Sodium Acrylate/Sodium Acryloyldimethyl Taurate Copolymer | 2 |
| Althaea Officinalis (Marsh-Mallow) Root Extract | 5 |
| Beta-Glucan | 3 |
| Aloe Barbadensis (Aloe) Leaf Extract | 3 |
| Sodium Hyaluronate | 3 |
| Allantoin | 2 |
| Ethylhexylglycerin | 0.5 |
| TOTAL | 100 |

Phase II of Scar Protocol was designed to treat the wound as it cycles between stages of healing. At this point, the incision has a lower risk of infection and requires adequate sustenance. This allows it to finish the healing process and create a smooth, discreet scar. Therefore, the Phase II moisturizing scar cream was formulated to supply fundamental nutrients and moisture to the scarring area. Phase II is also designed to begin treating the scar less like a wound and more like a scar. This requires keeping the skin around it clean and healthy. It also addresses the new layers of skin and tissue that have formed over the area. This treatment can lead to a smoother, less visible scar.

When Phase II is started, scar tissue and skin should have healed over the wound. At this stage, it is critical to keep the scar and skin healthy to prevent a rough and raised scar. Phase II’s scar cream formula uses powerful ingredients to support healthy healing. By delivering a carefully formulated blend of vitamins, amino acids, minerals, and proteins, use of Phase II in the early stages of healing ensures that the wound receives the proper nutrition at the right time.

Phase II has several notable ingredients, including allantoin, glycerin, and aloe which keeps the scar moisturized and provides nutrients deep into the skin. These Phase II ingredients have several properties that contribute to the efficacy of the second treatment cream and thus, the protocol.

Allantoin. Allantoin is a metabolic element found in many plants, animals, and other organisms. Common sources of allantoin include: comfrey, chamomile, wheat sprouts, and sugar beets. This ingredient is used in many moisturizers because it can treat dry, flaky skin. In addition to powerful moisturization qualities, allantoin can improve skin texture, provide nourishment for new collagen and skin, promote cell regeneration, and prevent loss of healthy skin cells. During Phase II, the allantoin works to produce healthy skin cells instead of unsightly scar tissue. Additionally, this effective moisturizing agent enhances the feel of the skin and promotes a smoother, less visible scar. Scar tissue, because it is different from skin tissue, often feels rougher and can sometimes appear raised. The allantoin encourages a smoother, flatter surgery scar.

Glycerin. Glycerin is a type of sugar derived from a variety of sources, including plant oils. Often used in the food industry as a sweetener and preservative, glycerin is also an FDA approved treatment for wounds. It contains natural components that ward off harmful microbes and viruses. Glycerin prevents infection, treats inflammation, and protects the incision, so that it can focus on healing instead of fighting off infections. Not only does glycerin provide moisture to the skin, it attracts moisture from the environment. Glycerin is a humectant, which are substances that attract and draw water deep into the application areas. Humectants can be found in lotions and food. Other natural humectants are: honey, egg yolks and whites, and molasses. Consequently, glycerin draws in nearby moisture and helps to nourish and moisturize below the surface. This will not only promote healthy healing, but also keeps the subject more comfortable during this stage of healing. This stage can be itchy and painful due to the formation of new tissue structures like skin and blood vessels.

### Phase III

Phase III includes a third scar treatment cream. Phase III starts around month four of the 12 month protocol. Phase III should begin immediately after completing Phase II and should be continued for about two to three months (or until the cream runs out). The third scar treatment cream is focused on smoothing the scar using a purified white snail mucin based product to help restore damaged cells and prevent discoloration. In combination with other natural ingredients, snail mucin improves the health of the skin, strengthens the forming skin and tissue structures, and promotes the skin cell turnover process. This two or three month regimen accounts for an important time in the healing process where the visibility of the scar begins to form and worsen after the initial healing of the wound. Starting treatment of the scar early can help dramatically reduce the appearance of the final scar that forms. The third treatment cream is formulated to include the ingredients shown in Table 4 below.

TABLE 4

Third Treatment Cream Ingredients (by total weight of composition)

| Ingredient | % by weight |
|---|---|
| Aqua (Deionized Water) | 45 |
| Glycerol | 5 |
| Snail Mucin Extract | 5 |
| Viola Tricolor (Wild Pansy) Extract | 3 |
| Aloe Barbadensis (Aloe) Leaf Juice | 8 |
| Butyrospermum Parkii (Shea Tree) | 4.5 |
| Oryza Sativa (Rice) Germ | 0.5 |
| Rosa Gallica (French Rose) Extract | 2 |
| Caprylic / Capric Triglyceride | 8 |
| Squalane | 1 |
| Betaine | 5 |
| Dimethicone | 3 |
| Xanthan Gum | 3 |
| Tocopherol (Vitamin E) | 5 |
| Laurocapram | 1.5 |
| Sodium Hyaluronate | 0.5 |
| TOTAL | 100 |

The final pigmentation of the scar has not yet settled by Phase III. Treating discoloration throughout the scarring process will encourage a less visible scar once fully healed. Scars tend to look darker than the surrounding skin. Therefore, Phase III gets an early start in treating this pigmentation issue to reduce its noticeability later on. During testing of the product, it was found that including pigmentation management at this phase of the scar treatment led to better results. When Phase III is applied, it should cover the entire scar and should be applied twice daily over two to three months.

At the stage when Phase III starts, the scar looks like a traditional surgical or wound scar: the incision should look mostly healed over, with some minor redness due to the formation of the new skin over the wound. It may look discolored and feel a little rougher than the rest of the skin. Protecting this new skin is important at this stage. Phase III uses a mix of ingredients including purified white snail mucin, oryza sativa, and rosa gallica that contain high amounts of vitamins and antioxidants to nurture the skin at this time. Use of the third scar treatment will comfortably resurface the skin and work to fade the pigment of the scar. The Phase III ingredients have several properties that contribute to the efficacy of the third treatment cream and thus, the protocol.

Purified Snail Mucin. The main ingredient of Phase III is purified snail mucin extract. Snail mucin is a highly hydrating ingredient that will keep the scar well-nourished throughout Phase III. Purified snail mucus was discovered to be beneficial in skin care by Chilean snail breeders in the 1980s. The breeders who worked with the snails everyday began to notice their hands becoming soft and supple. The mucus can be collected by placing snails on a net in a dark room and given the room to run free. Their slime drips through the net and into the collection containers. The mucus is then purified and filtered to remove any soil or contaminants that may be present. Originally used to hydrate the skin, recent science has shown that purified snail mucus may aid in wound healing. Among the beneficial components found naturally in purified snail mucin include: allantoin, zinc, copper peptides, vitamins A and E, among others. This means that this slime works double duty by keeping the skin soft and moisturized, while also aiding in collagen production and the healing of the incision. The purified white snail mucin uses its dynamic restorative properties to help rebuild the skin without the use of excessive scar tissue on the surface. Treating the scar at this stage can help it form with a smoother texture and less noticeable appearance.

Oryza Sativa. Oryza sativa is a natural substance derived from rice. Along with being one of the world’s staple crops, rice can provide many benefits to the skin when in certain forms. As it relates to the embodiments described herein, oryza sativa works to even out the skin tone and correct the discoloration of the forming scar. The fundamental component of oryza sativa that contributes to this fight against discoloration is gamma-oryzanol. Gamma-oryzanol is a mixture of fatty acids that occur naturally in rice and wheat bran. It can curb puffiness, inflammation, and dark under eye circles. These attributes also contribute to treatment of the scar. The ingredient works with purified snail mucus to reduce the coloration of the scar before it fully sets in.

Rosa Gallica. Rosa gallica, or French Rose, offers anti-inflammatory properties and has been known to reduce skin damage from UV sun damage. For scars not possible to keep covered, such as those on the face or neck, rosa gallica offers extra protection from the sun. This prevents the sun from damaging the scar or the surrounding skin, which can lead to a worsened appearance.

### Phase IV

Phase IV includes a fourth scar treatment cream, which may also be referred to as a removal cream. Phase IV starts around month six of the 12 month protocol. Phase IV should begin immediately after completing Phase III and should be continued for three or four months (or until the cream runs out). The Phase IV Removal Cream targets scar redness to reduce and calm the appearance of the scar. It also creates strong, healthy skin for a seamless and discreet scar. Using ingredients including tocopherol and salix alba, this cream works to disguise the scar into the skin. Additionally, it contains powerful bonding agents like coco oil that prepare it for the final phase. The fourth treatment cream is formulated to include the ingredients shown in Table 5 below.

TABLE 5

Fourth Treatment Cream Ingredients (by total weight of composition)

| Ingredient | % by weight |
|---|---|
| Aqua (Deionized Water) | 48.5 |
| Theobroma Cacao (Cocoa) Butter | 15 |
| Butyrospermum Parkii (Shea) Butter | 5 |
| Cocos Nucifera (Coconut) Oil | 8 |
| Glycerin | 5 |
| Tocopherol (Vitamin E) | 5 |
| Polydimethylsiloxane (Silicone) | 0.5 |
| Xanthan Gum | 5 |
| Salix Alba (White Willow) Bark Extract | 3 |
| Rosmarinus Officianalis (Rosemary) Leaf Extract | 3 |
| Azadirachta Indica (Neem) Seed Oil | 2 |
| TOTAL | 100 |

Phase IV encompasses a three-month treatment period, and is applied twice daily (ensuring that the cream covers the entire scar). The scar may also benefit from massaging in the Phase IV cream to better absorb it into the skin. Phase IV works to decrease the appearance of the scar by addressing the pigment.

When Phase IV is started, it is roughly six months into Scar Protocol. At this time, scar generation is still in the early stages of the healing process. The skin should be fully healed and the body in the middle of forming the scar. This scar formation process can last up to two years after the initial surgery or wound. Using the Phase IV cream at this time addresses the problem before it can fully form, giving the best chance at an optimal result. During this stage of healing, the skin likely looks fully healed and the pigment and the scar starts to become more apparent. Utilizing the specially designed removal cream, the focus of the Phase IV cream is to encourage the still forming scars to fade or soften the look of the scar, hinder scar tissue growth, and to provide nourishment and support to new and existing skin tissue.

Phase IV of the Scar Protocol does not aim simply to remove a scar but to prevent thick scarring from forming. It does this through a distinct blend of ingredients that help the new and existing skin tissue bond together to form smoother and stronger, overall decreasing the appearance of the scar.

Phase IV ingredients improve the health of the skin, stimulate collagen production, and encourage a cohesive bonding of the new skin. Phase IV treats the texture of the scar while adding the nutrients the skin and scar need. It also supports overall healing because it penetrates underneath the surface of the skin. Along with working below the skin, Phase IV protects the area from the environment and outside factors. Phase IV does this with ingredients such as tocopherol, salix alba, and coco oil.

Tocopherol.Tocopherol is a potent form of vitamin E sourced from vegetable oils like olive and soybean oil. This antioxidant helps protect cells from damage and moisturizes the skin to keep it soft and malleable. This makes it easier to treat, flatten, and decrease the appearance of the surgical scar. Tocopherol offers many benefits to the skin and scarring like moisturization, treatment of sun damaged skin, restoration of lost vitamin E, and fighting the signs of aging. Tocopherol is also important in that it creates a protective layer on the skin. This keeps the scar and skin safe from free radicals that could contaminate it like cigarette smoke, air pollutants, and UV rays from the sun. This is especially important for facial scars. In Phase IV of the scar protocol, tocopherol ensures that the skin remains moisturized, healthy, and protected from the elements. Additionally, tocopherol can restore lost vitamin E from aging and the damage to the skin caused by the incision or injury. This will support new skin structures and allow the body to focus on forming a non-descript scar.

Salix Alba.Salix alba, or white willow bark, contains several beneficial qualities that make it an important ingredient in the scar protocol. First, in addition to its ability to treat pain, inflammation, and fever, Salix alba is also known to reduce redness, provides exfoliation, and prevents skin irritation. All of these benefits help disguise the scar and reduce the difference between the surrounding skin and the scar. When it comes to cutting down redness, this will treat the discoloration caused by the scar. Additionally, salix alba contains tannins, which are organic substances found in many tree barks. Tannins act like a toner and work to shrink the skin and pores. This can reduce the outward appearance of the scar and can help firm up the look of the surgical area. The myriad of benefits that salix alba provides makes it the perfect ingredient for the scar protocol. With wound healing characteristics, white willow functions as a moisturizer, pain reliever, and healing agent.

Coconut Oil.Coconut oil is a natural treatment for everything from aiding weight loss to supporting soft and healthy hair. This is no exception when it comes to skin and scar treatment. Coconut oil creates a thick layer that locks in moisture. When used with other moisturizing agents, it can protect moisturizers from being broken down by environmental elements. Coconut oil also contains caprylic and lauric acids which can help even skin tone and can prevent harmful microbes from taking root in the area around the scar. While coconut oil provides many basic benefits for the skin, the main function that this tropical fruit oil serves to bind together new skin tissue. This lessens scarring by smoothing the skin and encouraging collagen production. Coconut oil serves an important binding agent for the skin and the scar protocol includes enough coconut oil to provide optimal benefit without clogging pores. Using coconut oil early in scar formation helps the new skin bond with the skin around the scar.

### Phase V

Phase V includes a fifth scar treatment cream. Phase V starts around month ten of the 12 month protocol. Phase V should begin after completing Phase IV and should be continued for two or three months (or until the cream runs out). The fifth scar treatment cream is a retinol cream. This final phase of the scar system provides the finishing touches the scar needs to look flat and smooth. In the previous phase (Phase IV), healthy bonds are created between the newly produced skin and the active skin cells. Phase V works to create a seamless transition between these bonds. This smooths the texture of the scar and the shift from unaffected skin to the scar. The Phase V formula encompasses powerful and potent ingredients. It wraps up the first year of healing and completes the scar protocol system. The fifth treatment cream is formulated to include the ingredients shown in Table 6 below.

TABLE 6

Fifth Treatment Cream Ingredients (by total weight of composition)

| Ingredient | % by weight |
|---|---|
| Aqua (Deionized Water) | 45.5 |
| Aloe Barbadensis (Aloe) Extract | 8 |
| Helianthus Annuus (Sunflower) Oil | 5 |
| Glyceryl Stearate | 0.5 |
| Stearic Acid | 8 |
| Cassia Angustifolia (Senna) Seed | 3 |
| Vegetable Glycerin | 2 |
| Retinol 2.5% | 2.5 |
| Simmondsia Chinensis (Jojoba) Oil | 5 |
| Camellia Sinensis (Green Tea) Leaf Extract | 5 |
| Polydimethylsiloxane (Silicone) | 0.5 |
| Resina Propoli (Beeswax Acid) | 0.5 |
| Butyrospermum Parkii (Shea) Butter | 5 |
| Panthenol (Vitamin B5) | 2 |
| Tocopherol (Vitamin E) | 2 |
| Xanthan Gum | 3 |
| Phospholipids (Sunflower Lecithin) | 0.5 |
| Pentylene Glycol | 0.5 |
| Polysorbate 20 | 0.5 |
| Potassium Sorbate | 0.5 |
| Ethylhexyl Glycerin | 0.5 |
| TOTAL | 100 |

The scar continues to form beneath the surface of the skin and needs specialized care. Phase V works to decrease scar formation, smooth the texture and look of the scar, and boost cell turnover rates. The application of Phase V follows the same instructions as the previous phase scar creams (twice daily) for the duration of Phase V. When Phase V begins, it will be 10 months into the 12-month scar protocol regime and will be continued for the final two or three months. The final phase is important to seeing results and inhibit the last stages of scar formation, because Phase V contains the most potent ingredients of the scar protocol system, including retinol to resurface the skin and tissue of the scar, which can lead to some minor and temporary dryness. The scar protocol includes ingredients to balance out any possible dryness. Some mild side effects of the retinol may also be noticed at first (e.g., light peeling or redness), though they should subside as the skin adjusts to the retinol.

While the scar will look healed by Phase V, it is still healing under the surface. Phase V of Scar Protocol, like previous phases, works both on the skin and deep under the surface. The cream addresses the outward appearance of the scar with more fervor than able to in the earlier phases. Moisturizing and nourishing ingredients continue to support healing. When the scar protocol and Phase V is finished, the scar is expected to be more faded and smooth than if the scar protocol had never been used. When Phase V is finished, the scar should have an improved appearance and feel smooth to the touch. Even if the skin is not completely done healing, Scar Protocol works to provide the least visible scar possible through the creation of new, healthy skin cells.

Phase V has several notable ingredients, including retinol (as noted above), helianthus, and jojoba. These Phase V ingredients have several properties that act to resurface the scar for a smoother feel, faded appearance, and contribute to the efficacy of the fifth treatment cream and thus, the protocol.

Retinol.Retinol is a derivative of vitamin A. All derivatives of Vitamin A are known as retinoids. Retinol is known for its powerful anti-aging benefits, but retinol can also dramatically improve the appearance and quality of the skin and scar. Retinol is found in wrinkle reducing products, skin serums, and night creams. Topical application of retinol (and other retinoids) increases cell growth factors, supports formation of new blood vessels, and boosts collagen production. This all contributes to healthier skin and a better looking scar. The science behind retinol is that it increases the rate the cells turnover. This means that the retinol removes dead skin and damaged cells, keeping the skin healthy and glowing. The continued elimination of non-active skin cells activates the body to make collagen, which aids in the healing process and naturally renews the skin of and surrounding the scar for a smoother appearance. Retinol can cause some uncomfortable side effects like skin irritation and peeling. However, including a minimal amount of retinol in combination with other soothing ingredients provides a balance to harness the powerful benefits of retinol for surgical scars, while keeping uncomfortable side effects to a minimum.

Helianthus.Helianthus annuus, or the common sunflower, is used in the form of an oil to protect and provide important nutrients to the skin. Sunflower oil contains zinc, calcium, iron, and a variety of vitamins including A, E, and K. This keeps the skin hydrated. Like other fruit and flower oils, it acts as the skin’s defense against free radicals and UV rays to keep damaging materials from penetrating the skin. When it comes specifically to scarring, sunflower oil contains Omega-6, also known as linoleic acid, which expedites the cell turnover process. Omega-6 is a fatty acid that helps the function of all bodily cells. As retinol continues healthy cell turnover, the sunflower oil will nourish the young cells and keep the skin in good condition.

Jojoba. Jojoba oil comes from the nut of the jojoba plant that lives in the deserts of the American Southwest. The plant can survive in harsh desert environments because it retains vitamins, minerals, and water. These properties can be transferred to the skin through the use of jojoba oil. On the surface, jojoba oil keeps the skin moisturized and protected. Like glycerin in Phase II, jojoba is a humectant that locks in moisture. It also helps counteract any side effects from retinol use and keeps Phase V moisturizing and pleasant. Jojoba oil has also been known to soothe sunburns and acne. In addition to serving as moisturizer and protectant from the elements, jojoba oil promotes collagen production, regulates sebum production, has anti-inflammatory properties, and aids the healing process. It is also hypo-allergenic and does not clog the pores. The versatility of jojoba oil and the comfort it provides makes it the perfect ingredient to supplement the hardy ingredients of Phase V.

### EXAMPLES

Several aspects of the present technology are set forth in the following examples:

1. A set of topical compositions, comprising:
a cleansing composition comprising polydimethylsiloxane (silicone) and Rosmarinus officinalis (rosemary) flower extract;
a first treatment composition comprising Aloe barbadensis (aloe or aloe vera) leaf juice, Macadamia ternifolia (macadamia) seed oil, and panthenol (vitamin B5);
a second treatment composition comprising Aloe barbadensis (aloe or aloe vera) leaf extract, sodium hyaluronate, and allantoin;
a third treatment composition comprising snail mucin extract;
a fourth treatment composition comprising tocopherol; and
a fifth treatment composition comprising retinol.

2. A kit comprising the set of topical compositions of example 1, further comprising a container that holds (i) a set of six containers that contain the cleansing composition, the first treatment composition, the second treatment composition, the third treatment composition, the fourth treatment composition, and the fifth treatment composition, and (ii) an instructional insert card.

3. A method for treating or preventing a scar or a wound, the method comprising:
applying a cleansing composition followed by a first treatment composition to a wound or scar at least once daily for a first period of time, wherein the cleansing composition comprises silicone and rosemary flower extract, and wherein the first treatment composition comprises aloe leaf juice, macadamia seed oil, and vitamin B5;
after the first period of time, applying a second treatment composition to the wound or scar at least once daily for a second period of time, wherein the second treatment composition comprises aloe leaf extract, sodium hyaluronate, and allantoin;
after the second period of time, applying a third treatment composition to the wound or scar at least once daily for a third period of time, wherein the third treatment composition comprises snail mucin extract;
after the third period of time, applying a fourth treatment composition to the wound or scar at least once daily for a fourth period of time, wherein the fourth treatment composition comprises tocopherol;
after the fourth period of time, applying a fifth treatment composition to the wound or scar at least once daily for a fifth period of time, wherein the fifth treatment composition comprises retinol.

4. The method of example 3, wherein the first period of time is about one month.

5. The method of example 3 or 4, wherein the second period of time is about 2 or 3 months.

6. The method of any one of examples 3 to 5, wherein the third period of time is about 2 or 3 months.

7. The method of any one of examples 3 to 6, wherein the fourth period of time is about 3 or 4 months.

8. The method of any one of examples 3 to 7, wherein the fifth period of time is about 2 or 3 months.

9. The method of any one of examples 3 to 8, wherein each of the treatment compositions are applied twice daily.

10. A scar treatment protocol, comprising:
a first phase comprising a cleanser composition and a first treatment composition that are applied to a wound or scar at least once daily for about a month, wherein the cleanser composition comprises silicone and rosemary flower extract and the first treatment composition comprises aloe leaf juice, macadamia seed oil, and vitamin B5;
a second phase comprising a second treatment composition that is applied to the wound or scar at least once daily for at least two months, wherein the second treatment composition comprises aloe leaf extract, sodium hyaluronate, and allantoin;
a third phase comprising a third treatment composition that is applied to the wound or scar at least once daily for at least two months, wherein the third treatment composition comprises snail mucin extract;
a fourth phase comprising a fourth treatment composition that is applied to the wound or scar at least once daily for at least three months, wherein the fourth treatment composition comprises tocopherol; and
a fifth phase comprising a fifth treatment composition that is applied to the wound or scar at least once daily for at least two months, wherein the fifth treatment composition comprises retinol.

11. A wound treatment protocol, comprising:
a first phase comprising a cleanser composition and a first treatment composition that are applied to a wound or scar at least once daily for about a month, wherein the cleanser composition comprises silicone and rosemary flower extract and the first treatment composition comprises aloe leaf juice, macadamia seed oil, and vitamin B5;
a second phase comprising a second treatment composition that is applied to the wound or scar at least once daily for at least two months, wherein the second treatment composition comprises aloe leaf extract, sodium hyaluronate, and allantoin;
a third phase comprising a third treatment composition that is applied to the wound or scar at least once daily for at least two months, wherein the third treatment composition comprises snail mucin extract;
a fourth phase comprising a fourth treatment composition that is applied to the wound or scar at least once daily for at least three months, wherein the fourth treatment composition comprises tocopherol; and
a fifth phase comprising a fifth treatment composition that is applied to the wound or scar at least once daily for at least two months, wherein the fifth treatment composition comprises retinol.

12. The method or treatment protocol of any one of examples 3 to 11, wherein treatment is started between about a week to about six months after obtaining or receiving the wound or scar

13. The method or treatment protocol of any one of examples 3 to 12, wherein the wound or scar is an incision, a laceration, or a sore.

14. The method or treatment protocol of any one of examples 3 to 13, wherein the application is topical

15. The collection of topical compositions, kit, method, or treatment protocol of example 1, wherein the silicone is present in the cleansing composition in an amount of about 5% by weight.

16. The collection of topical compositions, kit, method, or treatment protocol of any one of examples 1 to 15, wherein the rosemary flower extract is present in the cleansing composition in an amount of about 15% by weight.

17. The collection of topical compositions, kit, method, or treatment protocol of any one of examples 1 to 16, wherein the aloe leaf juice is present in the first treatment composition in an amount of about 15% by weight.

18. The collection of topical compositions, kit, method, or treatment protocol of any one of examples 1 to 17, wherein the macadamia seed oil is present in the first treatment composition in an amount of about 1.5% by weight.

19. The collection of topical compositions, kit, method, or treatment protocol of any one of examples 1 to 17, wherein panthenol (vitamin B5) is present in the first treatment composition in an amount of about 2% by weight.

20. The collection of topical compositions, kit, method, or treatment protocol of any one of examples 1 to 19, wherein aloe leaf extract is present in the second treatment composition in an amount of about 3% by weight.

21. The collection of topical compositions, kit, method, or treatment protocol of any one of examples 1 to 20, wherein Sodium Hyaluronate is present in the second treatment composition in an amount of about 3% by weight.

22. The collection of topical compositions, kit, method, or treatment protocol of any one of examples 1 to 21, wherein allantoin is present in the second treatment composition in an amount of about 2% by weight.

23. The collection of topical compositions, kit, method, or treatment protocol of any one of examples 1 to 22, wherein snail mucin extract is present in the third treatment composition in an amount of about 5% by weight.

24. The collection of topical compositions, kit, method, or treatment protocol of any one of examples 1 to 23, wherein tocopherol is present in the fourth cleansing composition in an amount of about 5% by weight.

25. The collection of topical compositions, kit, method, or treatment protocol of any one of examples 1 to 24, wherein retinol is supplied at a strength of 2.5% and is present in the fifth treatment composition in an amount of about 2.5% by weight.

26. The collection of topical compositions, kit, method, or treatment protocol of any one of examples 1 to 25, wherein the cleansing composition further comprises the contents of Table 1.

27. The collection of topical compositions, kit, method, or treatment protocol of any one of examples 1 to 26, wherein the first treatment composition further comprises the contents of Table 2.

28. The collection of topical compositions, kit, method, or treatment protocol of any one of examples 1 to 27, wherein the second treatment composition further comprises the contents of Table 3.

29. The collection of topical compositions, method, kit, or treatment protocol of any one of examples 1 to 28, wherein the third treatment composition further comprises the contents of Table 4.

30. The collection of topical compositions, method, kit, or treatment protocol of any one of examples 1 to 29, wherein the fourth treatment composition further comprises the contents of Table 5.

31. The collection of topical compositions, method, kit, or treatment protocol of any one of examples 1 to 30, wherein the fifth treatment composition further comprises the contents of Table 6.

### Conclusion

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments. For example, although this disclosure has been written to describe compositions that are generally described as being used for treating or preventing a scar, it should be appreciated that the disclosed compositions could be utilized for other suitable therapeutic purposes on various regions of the body.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A set of topical compositions, comprising:
a cleansing composition; and
at least one treatment composition comprising at least one component selected from the group consisting of Aloe barbadensis leaf juice, snail mucin extract, tocopherol, and retinol.

2. The set of topical compositions of claim 1, wherein:
the cleansing composition comprises polydimethylsiloxane (silicone) and Rosmarinus officinalis (rosemary) flower extract, and
the first treatment composition comprises Aloe barbadensis (aloe or aloe vera) leaf juice, Macadamia ternifolia (macadamia) seed oil, and panthenol (vitamin B5).

3. The set of topical compositions of claim 2, further comprising:
a second treatment composition which comprises Aloe barbadensis leaf extract, sodium hyaluronate, and allantoin;
a third treatment composition which comprises snail mucin extract;
a fourth treatment composition which comprises tocopherol; and
a fifth treatment composition which comprises retinol.

4. The set of topical compositions of claim 3, wherein:
the polydimethylsiloxane is present in the cleansing composition in an amount of about 5% by weight;
the rosemary flower extract in the cleansing composition is present in the cleansing composition in an amount of about 15% by weight;
the aloe leaf juice is present in the first treatment composition in an amount of about 15% by weight;
the macadamia seed oil is present in the first treatment composition in an amount of about 1.5% by weight;
the panthenol (vitamin B5) is present in the first treatment composition in an amount of about 2% by weight;
the aloe leaf extract is present in the second treatment composition in an amount of about 3% by weight;
the Sodium Hyaluronate is present in the second treatment composition in an amount of about 3% by weight;
the allantoin is present in the second treatment composition in an amount of about 2% by weight;
the snail mucin extract is present in the third treatment composition in an amount of about 5% by weight;
the tocopherol is present in the fourth treatment composition in an amount of about 5% by weight;
the retinol is supplied at a strength of 2.5% and is present in the fifth treatment composition in an amount of about 2.5% by weight;
or a combination thereof.

5. The set of topical compositions of claim 4, wherein the cleansing composition further comprises polydimethylsiloxane (silicone), decyl glucoside, Cocos Nucifera (Coconut) Oil, Europaea (Olive) Fruit Oil, Helianthus Annuus (Sunflower) Seed Oil, Aloe Barbadenis (Aloe) Leaf Juice, Citric Acid, Butyrospermum Parkii (Shea) Butter, Rosmarinus Officinalis (Rosemary) Oil, Rosmarinus Officinalis (Rosemary) Flower Extract, or any combination thereof.

6. The set of topical compositions of claim 5, wherein the first treatment composition further comprises Aloe Barbadensis (Aloe) Leaf Juice, Aqua (Deionized Water), Tocopheryl Acetate (Vitamin E), Helianthus Annuus (Sunflower) Seed Oil, Glyceryl Steswax, Potassium Stearate, Simmondsia Chinensis (Jojoba) Seed Oil, Butyrospermum Parkii (Shea) Butter, Calophyllum Inophyllum (Tamanu) Seed Oil, Macadamia Temifolia (Macadamia) Seed Oil, Aleurites Moluccana (Kukui) Seed Oil, Persea Gratissima (Avocado) Oil, Borago Officinalis (Borage) Seed Oil, Linum Usitatissimum (Flax) Seed Oil, Oenothera Biennis (Evening Primrose) Oil, Rosa Canina (Rosehip) Fruit Oil, Panthenol (Vitamin B5), Camellia Sinensis (Green Tea) Leaf Extract, Morinda Citrifolia (Noni) Extract, Passiflora Incarnata (Passionflower) Extract, Calendula Officinalis (Calendula) Extract, Chamomilla Recutita (Chamomile) Extract, Lavandula Angustifolia (Lavender) Extract, Arnica Montana (Arnica) Extract, Centella Asiatica (Gotu Kola) Extract, Equisetum Arvense (Horsetail) Extract, Geranium Maculatum (Wild Geranium) Extract, Taraxacum Officinale (Dandelion) Extract, Stearic Acid, Xanthan Gum, Sodium Carbomer, Pentylene Glycol, or any combination thereof.

7. The set of topical compositions of claim 6, wherein the second treatment composition further comprises Aqua (Deionized Water), Glycerin, Propylene Glycol, Ethylhexyl Palmitate (Palm Oil Derivate), Sorbitan Olivate, Caprylic/Capric Triglyceride, Paraffinum Liquidum, Squalane, Dimethicone, Isohexadecane, Hydrogenated Coco-glycerides (Coconut Oil), Polysorbate 60, Hydroxyethyl Acrylate/Sodium Acryloyldimethyl Taurate Copolymer, Sodium Acrylate/Sodium Acryloyldimethyl Taurate Copolymer, Althaea Officinalis (Marsh-Mallow) Root Extract Beta-Glucan, Aloe Barbadensis (Aloe) Leaf Extract, Sodium Hyaluronate, Allantoin, Ethylhexylglycerin, or any combination thereof.

8. The set of topical compositions of claim 7, wherein the third treatment composition further comprises Aqua (Deionized Water), Glycerol, Snail Mucin Extract, Viola Tricolor (Wild Pansy) Extract, Aloe Barbadensis (Aloe) Leaf Juice, Butyrospermum Parkii (Shea Tree), Oryza Sativa (Rice) Germ, Rosa Gallica (French Rose) Extract, Caprylic / Capric Triglyceride, Squalane, Betaine, Dimethicone, Xanthan Gum, Tocopherol (Vitamin E), Laurocapram, Sodium Hyaluronate, or any combination thereof.

9. The set of topical compositions of claim 8, wherein the fourth treatment composition further comprises the contents of Aqua (Deionized Water), Theobroma Cacao (Cocoa) Butter, Butyrospermum Parkii (Shea) Butter, Cocos Nucifera (Coconut) Oil, Glycerin, Tocopherol (Vitamin E), Polydimethylsiloxane (Silicone), Xanthan Gum, Salix Alba (White Willow) Bark Extract, Rosmarinus Officianalis (Rosemary) Leaf Extract, Azadirachta Indica (Neem) Seed Oil, or any combination thereof.

10. The set of topical compositions of claim 9, wherein the fifth treatment composition further comprises the contents of Aqua (Deionized Water), Aloe Barbadensis (Aloe) Extract, Helianthus Annuus (Sunflower) Oil, Glyceryl Stearate, Stearic Acid, Cassia Angustifolia (Senna) Seed, Vegetable Glycerin, Retinol, Simmondsia Chinensis (Jojoba) Oil, Camellia Sinensis (Green Tea) Leaf Extract, Polydimethylsiloxane (Silicone), Resina Propoli (Beeswax Acid), Butyrospermum Parkii (Shea) Butter, Panthenol (Vitamin B5), Tocopherol (Vitamin E), Xanthan Gum, Phospholipids (Sunflower Lecithin), Pentylene Glycol, Polysorbate 20, Potassium Sorbate, Ethylhexyl Glycerin, or any combination thereof.

11. A method for treating or preventing a scar or wound, the method comprising:
applying a cleansing composition followed by a first treatment composition to a wound or scar at least once daily for a first period of time, wherein the cleansing composition comprises silicone and rosemary flower extract, and wherein the first treatment composition comprises aloe leaf juice, macadamia seed oil, and vitamin B5;
after the first period of time, applying a second treatment composition to the wound or scar at least once daily for a second period of time, wherein the second treatment composition comprises aloe leaf extract, sodium hyaluronate, and allantoin;
after the second period of time, applying a third treatment composition to the wound or scar at least once daily for a third period of time, wherein the third treatment composition comprises snail mucin extract;
after the third period of time, applying a fourth treatment composition to the wound or scar at least once daily for a fourth period of time, wherein the fourth treatment composition comprises tocopherol; and
after the fourth period of time, applying a fifth treatment composition to the wound or scar at least once daily for a fifth period of time, wherein the fifth treatment composition comprises retinol.

12. The method of claim 11, wherein the first period of time is about a month.

13. The method of claim 11, wherein the second period of time is about 2 or 3 months.

14. The method of claim 11, wherein the third period of time is about 2 or 3 months.

15. The method of claim 11, wherein the fourth period of time is about 3 or 4 months.

16. The method of claim 11, wherein the fifth period of time is about 2 or 3 months.

17. The method of claim 11, wherein the first period of time begins about a week to about six months after the wound occurs or the scar begins to form.

18. The method of claim 11, wherein the cleansing composition and each of the treatment compositions are applied topically to the wound or the scar.

19. The method of claim 11, wherein each of the treatment compositions are applied twice daily.

20. A kit comprising the set of topical compositions of claim 1, further comprising a container that holds:
(i) a container for containing the cleansing composition,
(ii) a container for containing each treatment composition, and
(iii) an instructional insert card.
